(19) United States
(12) Patent Application Publication
(10) Pub. No.: US 2007/0110813 A1
(43) Pub. Date: May 17, 2007

(54) Polycation-polyanion complexes, compositions and methods of use thereof

(75) Inventors: Edward P. Ingenito, North Quincy, MA (US); Alexander Schwarz, Brookline, MA (US); Larry W. Tsai, Boston, MA (US)

Correspondence Address: FOLEY HOAG, LLP;PATENT GROUP, WORLD TRADE CENTER WEST, 155 SEAPORT BLVD, BOSTON, MA, 02110, US

(73) Assignee: AERIS THERAPEUTICS, INC., Wobum, MA (US)

(21) Appl. No.: 11/590,664

(22) Filed: Oct. 31, 2006

Related U.S. Application Data
(60) Provisional application No. 60/732,987, filed on Nov. 2, 2005.

(51) Int. Cl. A61K 38/48; A61K 9/14

(52) U.S. Cl. 424/488; 514/12; 424/94.64

(57) ABSTRACT

One aspect of the present invention relates to compositions and methods comprising polyelectrolyte molecules for treating patients who have certain diseases. Aspects of the invention relate to using certain polyelectrolyte compositions in therapy. According to the invention polyelectrolyte compositions may be used, for example, to slow or stop cell growth, kill cells (e.g., via necrotic or apoptotic pathways), promote fibrosis, or a combination thereof. In one aspect of the invention, certain toxic (e.g., cytotoxic) properties of polyelectrolytes are exploited for therapeutic purposes. In certain embodiments, compositions and methods of the invention are used to target polyelectrolyte toxicity to predetermined regions within a subject, while minimizing undesirable toxicity at other regions with the subject. In certain embodiments, the present invention relates to lung-volume-reduction therapy using a polyelectrolyte composition.

### RELATED APPLICATIONS

This application claims the benefit of priority to U.S. Provisional Patent Application Ser. No. 60/732,987, filed Nov. 2, 2005, which is hereby incorporated by reference in its entirety.

### BACKGROUND OF THE INVENTION

Emphysema is a common form of chronic obstructive pulmonary disease (COPD) that affects between 1.5 and 2 million Americans, and 3 to 4 times that number of patients worldwide. [American Thoracic Society Consensus Committee “Standards for the diagnosis and care of patients with chronic obstructive pulmonary disease,” Am. J. Resp. Crit. Care Med. 1995, 152, 78-83; and Pauwels, R., et al. “Global strategy for the diagnosis, management, and prevention of chronic obstructive pulmonary disease,” Am. J. Resp. Crit. Care Med. 2001, 163, 1256-1271.] It is characterized by destruction of the small airways and lung parenchyma due to the release of enzymes from inflammatory cells in response to inhaled toxins. [Stockley, R. “Neutrophils and protease/antiprotease imbalance,” Am. J. Resp. Crit. Care Med. 1999, 160, S49-S52.] Although this inflammatory process is usually initiated by cigarette smoking, once emphysema reaches an advanced stage, it tends to progress in an unrelenting fashion, even in the absence of continued smoking. [Rutgers, S. R., et al. “Ongoing airway inflammation inpatients with COPD who do not currently smoke,” Thorax 2000, 55, 12-18.]

The class of enzymes that are responsible for producing tissue damage in emphysema are known as proteases. These enzymes are synthesized by inflammatory cells within the body and, when released, they act to degrade the collagen and elastin fibers which provide mechanical integrity and elasticity to the lung. [Jeffery, P. “Structural and inflammatory changes in COPD: a comparison with asthma,” Thorax 1998, 53, 129-136.] The structural changes that result from the action of these enzymes are irreversible, cumulative, and are associated with loss of lung function that eventually leaves patients with limited respiratory reserve and reduced functional capacity. [Spencer, S. et al. “Health status deterioration inpatients with chronic obstructive pulmonary disease,” Am. J. Resp. Crit. Care Med. 2001, 163, 122-128; and Moy, M. L., et al. “Health-related quality of life improves following pulmonary rehabilitation and lung volume reduction surgery,” Chest 1999, 115, 383-389.]

In contrast to other common forms of COPD, such as asthma and chronic bronchitis for which effective medical treatments exist, conventional medical treatment is of limited value in patients with emphysema. Although emphysema, asthma, and chronic bronchitis each cause chronic airflow obstruction, limit exercise capacity, and cause shortness of breath, the site and nature of the abnormalities in asthma and chronic bronchitis are fundamentally different from those of emphysema. In asthma and chronic bronchitis, airflow limitation is caused by airway narrowing due to smooth muscle constriction and mucus hyper-secretion. Pharmacologic agents that relax airway smooth muscle and loosen accumulated secretions are effective at improving breathing function and relieving symptoms. Agents that act in this way include beta-agonist and anti-cholinergic inhalers, oral theophylline preparations, leukotriene antagonists, steroids, and mucolytic drugs.

In contrast, airflow limitation in emphysema is not primarily due to airway narrowing or obstruction, but rather to loss of elastic recoil pressure as a consequence of tissue destruction. Loss of recoil pressure compromises the ability to exhale fully, and leads to hyper-inflation and gas trapping. Although bronchodilators, anti-inflammatory agents, and mucolytic agents are frequently prescribed for patients with emphysema, they are generally of limited utility since they are intended primarily for obstruction caused by airway disease. They do nothing to address the loss of elastic recoil that is principally responsible for airflow limitation in emphysema. [Barnes, P. “Chronic Obstructive Pulmonary Disease,” N. Engl. J. Med. 2000, 343(4), 269-280.]

While pharmacologic treatments for advanced emphysema have been disappointing, a non-medical treatment of emphysema has recently emerged, which has demonstrated clinical efficacy. This treatment is lung volume reduction surgery (LVRS). [Flaherty, K. R. and F J. Martinez “Lung volume reduction surgery for emphysema,” Clin. Chest Med. 2000, 21(4), 819-48.]

LVRS was originally proposed in the late 1950s by Dr. Otto Brantigan as a surgical remedy for emphysema. The concept arose from clinical observations which suggested that in emphysema the lung was “too large” for the rigid chest cavity, and that resection of lung tissue represented the best method of treatment since it would reduce lung size, allowing it to fit and function better within the chest. Initial experiences with LVRS confirmed that many patients benefited symptomatically and functionally from the procedure. Unfortunately, failure to provide objective outcome measures of improvement, coupled with a 16% operative mortality, led to the initial abandonment of LVRS.

LVRS was accepted for general clinical application in 1994 through the efforts of Dr. Joel Cooper, who applied more stringent pre-operative evaluation criteria and modem post-operative management schemes to emphysema patients. [Cooper, J. D., et al. “Bilateral pneumonectomy for chornic obstructive pulmonary disease,” J. Thorac. Cardiovasc. Surg. 1995, 109, 106-119.] Cooper reported dramatic improvements in lung function and exercise capacity in a cohort of 20 patients with advanced emphysema who had undergone LVRS. There were no deaths at 90-day follow-up, and physiological and functional improvements were markedly better than had been achieved with medical therapy alone.

While less dramatic benefits have been reported by most other centers, LVRS has nevertheless proven to be effective for improving respiratory function and exercise capacity, relieving disabling symptoms of dyspnea, and improving quality of life in patients with advanced emphysema. [Gelb, A. F., et al. “Mechanism of short-term improvement in lung function after emphysema resection,” Am. J. Respir. Crit. Care Med. 1996, 154, 945-51; Gelb, A. F., et al. “Serial lung function and elastic recoil 2 years after lung volume reduction surgery for emphysema,” Chest 1998, 113(6), 1497-506; Criner, G. and G. E. D'Alonzo, Jr., “Lung volume reduction surgery: finding its role in the treatment of patients with severe COPD,” J. Am. Osteopath. Assoc. 1998, 98(7), 371; Brenner, M., et al. “Lung volume reduction surgery for emphysema,” Chest 1996, 110(1), 205-18; and Ingenito, E. P., et al. “Relationship between preoperative inspiratory lung resistance and the outcome of lung-volume-reduction surgery for emphysema,” N. Engl. J. Med. 1998, 338, 1181-1185.] The benefits of volume reduction have been confirmed in numerous cohort studies, several recently-completed small randomized clinical trials, and the National Emphysema Treatment Trial (NETT). [Goodnight-White, S., et al. “Prospective randomized controlled trial comparing bilateral volume reduction surgery to medical therapy alone inpatients with severe emphysema,” Chest 2000, 118 (Suppl 4), 1028; Geddes, D., et al. “L-effects of lung volume reduction surgery inpatients with emphysema,” N. Eng. J. Med. 2000, 343, 239-245; Pompeo, E., et al. “Reduction pneumoplasty versus respiratory rehabilitation in severe emphysema: a randomized study,” Ann. Thorac. Surg. 2000, 2000(70), 948-954; and Fishman, A., et al. “A randomized trial comparing lung-volume-reduction surgery with medical therapy for severe emphysema,” N. Eng. J. Med. 2003, 348(21): 2059-73.] On average, 75-80% of patients have experienced a beneficial clinical response to LVRS (generally defined as a 12% or greater improvement in FEV, at 3 month follow-up). The peak responses generally occur at between 3 and 6 months post-operatively, and improvement has lasted several years. [Cooper, J. D. and S. S. Lefrak “Lung-reduction surgery: 5 years on,” Lancet 1999, 353 (Suppl 1), 26-27; and Gelb, A. F., et al. “Lung function 4 years after lung volume reduction surgery for emphysema,” Chest 1999, 116(6), 1608-15.] Results from NETT have further shown that in a subset of patients with emphysema, specifically those with upper lobe disease and reduced exercise capacity, mortality at 29 months is reduced.

Collectively, these data indicate that LVRS improves quality of life and exercise capacity in many patients, and reduces mortality in a smaller fraction of patients, with advanced emphysema. Unfortunately, NETT also demonstrated that the procedure is very expensive when considered in terms of Quality Adjusted Life Year outcomes, and confirmed that LVRS is associated with a 5-6% 90 day mortality. [Chatila, W., S. Furukawa, and G. J. Criner, “Acute respiratory failure after lung volume reduction surgery,” Am. J. Respir. Crit. Care Med. 2000, 162, 1292-6; Cordova, F. C. and G. J. Criner, “Surgery for chronic obstructive pulmonary disease: the place for lung volume reduction and transplantation,” Curr. Opin. Pulm. Med. 2001, 7(2), 93-104; Swanson, S. J., et al. “No-cut thoracoscopic lung placation: a new technique for lung volume reduction surgery,” J. Am. Coll. Surg. 1997, 185(1), 25-32; Sema, D. L., et al. “Survival after unilateral versus bilateral lung volume reduction surgery for emphysema,” J. Thorac. Cardiovasc. Surg. 1999, 118(6), 1101-9; and Fishman, A., et al. “A randomized trial comparing lung-volume-reduction surgery with medical therapy for severe emphysema,” N. Engl. J. Med. 2003, 348(21), 2059-73.] In addition, morbidity following LVRS is common (40-50%) and includes a high incidence of prolonged post-operative air-leaks, respiratory failure, pneumonia, cardiac arrhythmias, and gastrointestinal complications. Less invasive and less expensive alternatives that could produce the same physiological effect are desirable.

A hydrogel-based system for achieving lung volume reduction has been developed and tested, and its effectiveness confirmed in both healthy sheep, and sheep with experimental emphysema. [Ingenito, E. P., et al. “Bronchoscopic Lung Volume Reduction Using Tissue Engineering Principles,” Am. J. Respir. Crit. Care Med. 2003, 167, 771-778.] This system uses a rapidly-polymerizing, fibrin-based hydrogel that can be delivered through a dual lumen catheter into the lung using a bronchoscope. The fibrin-based system effectively blocks collateral ventilation, inhibits surfactant function to promote collapse, and initiates a remodeling process that proceeds over a 4-6 week period. Treatment results in consistent, effective lung volume reduction. These studies have confirmed the safety and effectiveness of using fibrin-based hydrogels in the lung to achieve volume reduction therapy.

Sclerotherapy, a mechanism by which lung volume reduction may be achieved, is the injection of a chemical irritant (sclerosing agent) into a particular body lumen (e.g. a blood vessel or fallopian tube) to produce inflammation, a proliferation of connective tissue (i.e., fibrosis), and eventual obliteration of the lumen. Typical sclerosing agents include detergents, osmotic agents, and chemical irritants. Detergents such as sodium tetradecyl sulfate (Sotradecol), polidocanol (Aethoxysclerol), sodium morrhuate (Scleromate), and ethanolamine Oleate (Ethamolin), disrupt vein cellular membrane. Osmotic agents, such as hypertonic sodium chloride solution and sodium chloride solution with dextrose (Sclerodex), damage the cell by shifting the water balance. Chemical irritants, such as chromated glycerin (Sclermo), peroxides and polyiodinated iodine, damage the cell wall. Furthermore, talc can also be used in the lung (e.g., pleurodesis) as a sclerosing agent. Ethanol and acetic acid are used in bloodvessels as sclerosing agents. However, there remains a need in the art for effective, localized sclerotherapy compositions and methods. Such compositions and methods are disclosed herein.

### SUMMARY OF THE INVENTION

Certain aspects of the invention relate to using certain polyelectrolyte compositions in therapy. According to the invention polyelectrolyte compositions may be used, for example, to slow or stop cell growth, kill cells (e.g., via necrotic or apoptotic pathways), promote fibrosis, or a combination thereof. In one aspect of the invention, certain toxic (e.g., cytotoxic) properties of polyelectrolytes are exploited for therapeutic purposes. In certain embodiments, compositions and methods of the invention are used to target polyelectrolyte toxicity to predetermined regions within a subject, while minimizing undesirable toxicity at other regions with the subject.

According to the invention a subject may be a mammal. For example a subject may be a human, a pet, a domestic animal, a farm animal. In certain embodiments, a subject may be a dog, cat, horse, sheep, goat, primate, cow, pig, rat, mouse, or other animal.

A disease that may be treated may be any condition where abnormal cell growth and/or proliferation is undesirable. A therapy may include preventing further growth or proliferation or killing diseased cells or tissue. In other embodiments, a disease that may be treated may include any condition where fibrosis (e.g., scarring) may be useful. For example, certain conditions associated with abnormal tissue mechanical properties (e.g., emphysema) may be treated by promoting scarring. Finally, scarring also may be therapeutic under conditions where wound healing, tissue-tissue binding, and/or tissue-implant binding are helpful.

In some embodiments of the invention, a polycation may be provided in combination with one or more additional compounds that reduce the toxic (e.g., cytotoxic) properties of the cation while retaining sufficient activity to inhibit cell growth, kill cells, and/or promote fibrosis. In certain embodiments, a polycation may be complexed with a counterion (e.g., a polyanion) that counterbalances the charge of the polycation. Accordingly, in some embodiments, a polycation complex with a reduced net positive charge may be used in therapy.

In some aspects of the invention, a polycation may be provided in a gel (e.g., a hydrogel) or other immobilizing preparation (cream, matrix, etc.) to reduce its general toxic side-effects when administered to a subject. In some embodiments, the immobilizing preparation provides for delayed release of a therapeutic polycation.

It should be appreciated that compositions of the invention also may include one or more additional compounds (e.g., therapeutic compound(s), stabilizing compound(s), antibiotic(s), growth factor(s), etc.), buffers, salts, surfactants, anti-surfactants, lipids, excipients, and/or other suitable compounds. In certain embodiments, a composition of the invention may be sterilized. As described herein formulations of the invention may be used to reduce the number of positive charges on a polycation (to reduce the strength of certain toxic properties) while still retaining a threshold number of positive charges required to retain certain toxic or other properties that may be useful in therapy without causing excessive toxic side-effects. In some embodiments, the number of positive charges on a polycation may be reduced by complexing a polycation with an anion (e.g., a polyanion), by using certain salt or pH conditions that reduce the number of positive charges, by modifying the polycation to reduce the number of positive charges, and/or by using any other suitable technique for reducing or countering the number of positive charges on the polycation.

In certain embodiments, compositions of the invention may be used to promote one or more of the following responses when contacted to a tissue in a body: sclerosis (hardening of tissue), fibrosis (excess fibrous connective tissue), wound healing, tissue sealing, local microvascular thrombosis (blood clot), cellular necrosis or apoptosis (cell death), tumor regression, cell lysis, or any combination thereof.

Other advantages and novel features of the present invention will become apparent from the following detailed description of various non-limiting embodiments of the invention.

### BRIEF DESCRIPTION OF THE FIGURES

FIG. 1 depicts a tabulation of the results of in-vivo fibrin-gel experiments with polylysine and chondroitin sulfate. A “*” indicates that systemic heparin was administered (see group 7).

FIG. 2 depicts coronal CT images at baseline [A] and 6 weeks post treatment [B] in a patient receiving polylysine/chondroitin sulfate precipitate, delivered in a fibrin hydrogel to produce local tissue injury and lung volume reduction for treatment of emphysema. See example 10 in the Exemplification.

### DETAILED DESCRIPTION OF THE INVENTION

Certain aspects of the present invention relate to compositions and methods for treating patients who have certain diseases, and more specifically, to compositions and methods comprising one or more polycations (e.g., 1, 2, 3, 4, 5, 6, 7, 8, 9, 10) for treating patients who have certain diseases. In some cases, the disease can be treated by administering a composition comprising a polycation to induce a certain response (e.g., sclerosis and fibrosis) within a targeted region of the body. Compositions of polycations can vary depending on the particular response desired. In certain embodiments, it is desirable to administer a polycationic composition in a localized region within the body. Thus, the polycationic composition may be administered in a particular form (e.g., a gel) to induce local delivery of therapeutic agent.

Some polycations may be toxic to cells at certain concentrations, causing scarring, fibrosis, and other typically-undesired physiological responses in the body. If these polycations are administered controllably and locally to certain diseased regions of the body, however, the physiological response induced by the polycations may be therapeutically beneficial. For instance, polylysine may cause scarring and general toxicity (e.g., renal toxicity) when administered to patients. However, according to certain aspects of the invention, patients with certain conditions may be treated by causing damage such as scarring in a diseased region and polycation(s) can be beneficial and may cause a reversal of symptoms, as discussed in more detail below. Scarring can be induced in specific diseased regions of the body by administering compositions comprising polycations. The composition is preferably administered locally to avoid detrimental effects to other non-diseased regions of the body. In some embodiments, a polycation is complexed with a polyanion to reduce toxicity while retaining beneficial therapeutic effects described herein.

One aspect of the invention relates to compositions comprising polycations in amounts that may be toxic to certain diseased regions of the body, but which are provided in a therapeutic complex that is non-toxic, but can cause therapeutic effects in the diseased region. In certain embodiments, a complexed polycation retains a net positive charge. However, the net positive charge is lower than the net positive charge of the non-complexed polycation.

One aspect of the invention relates to compositions comprising polycations in amounts that may be toxic to certain diseased regions of the body, but which are provided in a form that results in release of the polycations in amounts that are non-toxic, and thereby cause therapeutic effects in the diseased region.

Another aspect of the invention relates to therapeutic uses of compositions comprising polycations to induce a certain response within a mammalian body. Such a response may include sclerosis, fibrosis, would healing, tissue sealing, localized microvascular thrombosis, cellular necrosis, and others, as described in more detail below.

The present invention also relates to treatment of certain medical conditions using compositions comprising polycations. In one aspect, a polycationic composition is used to treat emphysema (a chronic obstructive pulmonary disease (COPD)) by promoting localized fibrosis of diseased areas of the lung. In some cases, localized fibrosis is a means for achieving lung volume reduction (LVR).

In one embodiment, a polycationic composition is administered in a suitable form (e.g., in a gel, solution, or suspension) to a targeted diseased region of the lung. The polycationic composition may act as a cell-disrupting composition in some cases. In one particular embodiment, polycations are controllably released from a gel in an effective amount to cause damage to epithelial cells in the diseased region. Eliminating the epithelial barrier in a targeted area of the lung, in whole or in part, has been shown to improve the efficacy of lung volume reduction (e.g., BLVR). While it may seem counterintuitive that respiratory function would be improved by damaging or removing part of the lung, excising over-distended tissue (as seen in patients with heterogeneous emphysema) allows adjacent regions of the lung that are more normal to expand. In turn, this expansion allows for improved recoil and gas exchange. Even patients with homogeneous emphysema benefit from LVR because resection of abnormal lung results in overall reduction in lung volumes, an increase in elastic recoil pressures, and a shift in the static compliance curve towards normal [Hoppin, Am. J. Resp. Crit. Care Med. 1997, 155, 520-525].

According to aspects of the invention, a variety of polycations may be used, including but not limited to poly-L-lysine (PLL), poly-1-arginine, poly-ornithine, poly-ethylamine, and others, as discussed below. A variety of concentrations may be used (e.g., from 0.1% to 5.0%, or about 0.5%, or about 1%, or about 2%). Higher or lower concentrations also may be used depending on the potency of the polycation. It should be appreciated that different polycations may have different potencies. Polycation compositions of the invention may be used for other therapeutic applications as described herein.

Definitions

For convenience, before further description of the present invention, certain terms employed in the specification, examples, and appended claims are collected here. These definitions should be read in light of the remainder of the disclosure and as understood by a person of skill in the art.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising” “including,” “carrying” “having,” “containing,” “involving “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The term “amino acid” is intended to embrace all compounds, whether natural or synthetic, which include both an amino functionality and an acid functionality, including amino acid analogues and derivatives. In certain embodiments, the amino acids contemplated in the present invention are those naturally occurring amino acids found in proteins, or the naturally occurring anabolic or catabolic products of such amino acids, which contain amino and carboxyl groups.

Naturally occurring amino acids are identified throughout by the conventional three-letter and/or one-letter abbreviations, corresponding to the trivial name of the amino acid, in accordance with the following list: Alanine (Ala), Arginine (Arg), Asparagine (Asn), Aspartic acid (Asp), Cysteine (Cys), Glutamic acid (Glu), Glutamine (Gln), Glycine (Gly), Histidine (His), Isoleucine (Ile), Leucine (Leu), Lysine (Lys), Methionine (Met), Phenylalanine (Phe), Proline (Pro), Serine (Ser), Threonine (Thr), Tryptophan (Trp), Tyrosine (Tyr), and Valine (Val). The abbreviations are accepted in the peptide art and are recommended by the IUPAC-IUB commission in biochemical nomenclature.

The term “amino acid” further includes analogues, derivatives, and congeners of any specific amino acid referred to herein, as well as C-terminal or N-terminal protected amino acid derivatives (e.g., modified with an N-terminal or C-terminal protecting group).

The term “peptide” or “poly(amino acid)” as used herein, refers to a sequence of amino acid residues linked together by peptide bonds or by modified peptide bonds. These terms are intended to encompass peptide analogues, peptide derivatives, peptidomimetics and peptide variants. The term “peptide” or “poly(amino acid)” is understood to include peptides of any length.

The term “peptide analogue,” as used herein, refers to a peptide comprising one or more non-naturally occurring amino acid. Examples of non-naturally occurring amino acids include, but are not limited to, D-amino acids (i.e., an amino acid of an opposite chirality to the naturally occurring form), N-α-methyl amino acids, C-α-methyl amino acids, β-methyl amino acids, β-alanine (β-Ala), norvaline (Nva), norleucine (Nle), 4-aminobutyric acid (γ-Abu), 2-aminoisobutyric acid (Aib), 6-aminohexanoic acid (ε-Ahx), omithine (orn), hydroxyproline (Hyp), sarcosine, citrulline, cysteic acid, cyclohexylalanine, α-amino isobutyric acid, t-butylglycine, t-butylalanine, 3-aminopropionic acid, 2,3-diaminopropionic acid (2,3-diaP), D- or L-phenylglycine, D- or L-2-naphthylalanine (2-Nal), 1,2,3,4-tetrahydroisoquinoline-3-carboxylic acid (Tic), D- or L-2-thienylalanine (Thi), D- or L-3-thienylalanine, D- or L-1-, 2-, 3- or 4-pyrenylalanine, D- or L-(2-pyridinyl)-alanine, D- or L-(3-pyridinyl)-alanine, D- or L-(2-pyrazinyl)-alanine, D- or L-(4-isopropyl)-phenylglycine, D-(trifluoromethyl)-phenylglycine, D-(trifluoromethyl)-phenylalanine, D-p-fluorophenylalanine, D- or L-p-biphenylalanine, D- or L-p-methoxybiphenylalanine, methionine sulphoxide (MSO) and homoarginine (Har). Other examples include D- or L-2-indole(alkyl)alanines and D- or L-alkylalanines, wherein alkyl is substituted or unsubstituted methyl, ethyl, propyl, hexyl, butyl, pentyl, isopropyl, iso-butyl, or iso-pentyl, and phosphono- or sulfated (e.g., -SO₃H) non-carboxylate amino acids.

Other examples of non-naturally occurring amino acids include 3-(2-chlorophenyl)-alanine, 3-chloro-phenylalanine, 4-chloro-phenylalanine, 2-fluoro-phenylalanine, 3-fluoro-phenylalanine, 4-fluoro-phenylalanine, 2-bromo-phenylalanine, 3-bromo-phenylalanine, 4-bromo-phenylalanine, homophenylalanine, 2-methyl-phenylalanine, 3-methyl-phenylalanine, 4-methyl-phenylalanine, 2,4-dimethyl-phenylalanine, 2-nitro-phenylalanine, 3-nitro-phenylalanine, 4-nitro-phenylalanine, 2,4-dinitro-phenylalanine, 1,2,3,4-Tetrahydroisoquinoline-3-carboxylic acid, 1,2,3,4-tetrahydronorharman-3-carboxylic acid, 1-naphthylalanine, 2-naphthylalanine, pentafluorophenylalanine, 2,4-dichloro-phenylalanine, 3,4-dichloro-phenylalanine, 3,4-difluoro-phenylalanine, 3,5-difluoro-phenylalanine, 2,4,5-trifluoro-phenylalanine, 2-trifluoromethyl-phenylalanine, 3-trifluoromethyl-phenylalanine, 4-trifluoromethyl-phenylalanine, 2-cyano-phenyalanine, 3-cyano-phenyalanine, 4-cyano-phenyalanine, 2-iodo-phenyalanine, 3-iodo-phenyalanine, 4-iodo-phenyalanine, 4-methoxyphenylalanine, 2-aminomethyl-phenylalanine, 3-aminomethyl-phenylalanine, 4-aminomethyl-phenylalanine, 2-carbamoyl-phenylalanine, 3-carbamoyl-phenylalanine, 4-carbamoyl-phenylalanine, m-tyrosine, 4-amino-phenylalanine, styrylalanine, 2-amino-5-phenyl-pentanoic acid, 9-anthrylalanine, 4-tert-butyl-phenylalanine, 3,3-diphenylalanine, 4,4′-diphenylalanine, benzoylphenylalanine, α-methyl-phenylalanine, α-methyl-4-fluoro-phenylalanine, 4-thiazolylalanine, 3-benzothienylalanine, 2-thienylalanine, 2-(5-bromothienyl)-alanine, 3-thienylalanine, 2-furylalanine, 2-pyridylalanine, 3-pyridylalanine, 4-pyridylalanine, 2,3-diaminopropionic acid, 2,4-diaminobutyric acid, allylglycine, 2-amino-4-bromo-4-pentenoic acid, propargylglycine, 4-aminocyclopent-2-enecarboxylic acid, 3-aminocyclopentanecarboxylic acid, 7-amino-heptanoic acid, dipropylglycine, pipecolic acid, azetidine-3-carboxylic acid, cyclopropylglycine, cyclopropylalanine, 2-methoxy-phenylglycine, 2-thienylglycine, 3-thienylglycine, α-benzyl-proline, α-(2-fluoro-benzyl)-proline, α-(3-fluoro-benzyl)-proline, α-(4-fluoro-benzyl)-proline, α-(2-chloro-benzyl)-proline, α-(3-chloro-benzyl)-proline, α-(4-chloro-benzyl)-proline, α-(2-bromo-benzyl)-proline, α-(3-bromo-benzyl)-proline, α-(4-bromo-benzyl)-proline, α-phenethyl-proline, α-(2-methyl-benzyl)-proline, α-(3-methyl-benzyl)-proline, α-(4-methyl-benzyl)-proline, α-(2-nitro-benzyl)-proline, α-(3-nitro-benzyl)-proline, α-(4-nitro-benzyl)-proline, α-(1-naphthalenylmethyl)-proline, α-(2-naphthalenylmethyl)-proline, α-(2,4-dichloro-benzyl)-proline, α-(3,4-dichloro-benzyl)-proline, α-(3,4-difluoro-benzyl)-proline, α-(2-trifluoromethyl-benzyl)-proline, α-(3-trifluoromethyl-benzyl)-proline, α-(4-trifluoromethyl-benzyl)-proline, α-(2-cyano-benzyl)-proline, α-(3-cyano-benzyl)-proline, α-(4-cyano-benzyl)-proline, α-(2-iodo-benzyl)-proline, α-(3-iodo-benzyl)-proline, α-(4-iodo-benzyl)-proline, α-(3-phenyl-allyl)-proline, α-(3-phenyl-propyl)-proline, α-(4-tert-butyl-benzyl)-proline, α-benzhydryl-proline, α-(4-biphenylmethyl)-proline, α-(4-thiazolylmethyl)-proline, α-(3-benzo[b]thiophenylmethyl)-proline, α-(2-thiophenylmethyl)-proline, α-(5-bromo-2-thiophenylmethyl)-proline, α-(3-thiophenylmethyl)-proline, α-(2-furanylmethyl)-proline, α-(2-pyridinylmethyl)-proline, α-(3-pyridinylmethyl)-proline, α-(4-pyridinylmethyl)-proline, α-allyl-proline, α-propynyl-proline, γ-benzyl-proline, γ-(2-fluoro-benzyl)-proline, γ-(3-fluoro-benzyl)-proline, γ-(4-fluoro-benzyl)-proline, γ-(2-chloro-benzyl)-proline, γ-(3-chloro-benzyl)-proline, γ-(4-chloro-benzyl)-proline, γ-(2-bromo-benzyl)-proline, γ-(3-bromo-benzyl)-proline, γ-(4-bromo-benzyl)-proline, γ-(2-methyl-benzyl)-proline, γ-(3-methyl-benzyl)-proline, γ-(4-methyl-benzyl)-proline, γ-(2-nitro-benzyl)-proline, γ-(3-nitro-benzyl)-proline, γ-(4-nitro-benzyl)-proline, γ-(1-naphthalenylmethyl)-proline, γ-(2-naphthalenylmethyl)-proline, γ-(2,4-dichloro-benzyl)-proline, γ-(3,4-dichloro-benzyl)-proline, γ-(3,4-difluoro-benzyl)-proline, γ-(2-trifluoromethyl-benzyl)-proline, γ-(3-trifluoromethyl-benzyl)-proline, γ-(4-trifluoromethyl-benzyl)-proline, γ-(2-cyano-benzyl)-proline, γ-(3-cyano-benzyl)-proline, γ-(4-cyano-benzyl)-proline, γ-(2-iodo-benzyl)-proline, γ-(3-iodo-benzyl)-proline, γ-(4-iodo-benzyl)-proline, γ-(3-phenyl-allyl-benzyl)-proline, γ-(3-phenyl-propyl-benzyl)-proline, γ-(4-tert-butyl-benzyl)-proline, γ-benzhydryl-proline, γ-(4-biphenylmethyl)-proline, γ-(4-thiazolylmethyl)-proline, γ-(3-benzothioienylmethyl)-proline, γ-(2-thienylmethyl)-proline, γ-(3-thienylmethyl)-proline, γ-(2-fiuranylmethyl)-proline, γ-(2-pyridinylmethyl)-proline, γ-(3-pyridinylmethyl)-proline, γ-(4-pyridinylmethyl)-proline, γ-allyl-proline, γ-propynyl-proline, trans-4-phenyl-pyrrolidine-3-carboxylic acid, trans-4-(2-fluoro-phenyl)-pyrrolidine-3-carboxylic acid, trans-4-(3-fluoro-phenyl)-pyrrolidine-3-carboxylic acid, trans-4-(4-fluoro-phenyl)-pyrrolidine-3-carboxylic acid, trans-4-(2-chloro-phenyl)-pyrrolidine-3-carboxylic acid, trans-4-(3-chloro-phenyl)-pyrrolidine-3-carboxylic acid, trans-4-(4-chloro-phenyl)-pyrrolidine-3-carboxylic acid, trans-4-(2-bromo-phenyl)-pyrrolidine-3-carboxylic acid, trans-4-(3-bromo-phenyl)-pyrrolidine-3-carboxylic acid, trans-4-(4-bromo-phenyl)-pyrrolidine-3-carboxylic acid, trans-4-(2-methyl-phenyl)-pyrrolidine-3-carboxylic acid, trans-4-(3-methyl-phenyl)-pyrrolidine-3-carboxylic acid, trans-4-(4-methyl-phenyl)-pyrrolidine-3-carboxylic acid, trans-4-(2-nitro-phenyl)-pyrrolidine-3-carboxylic acid, trans-4-(3-nitro-phenyl)-pyrrolidine-3-carboxylic acid, trans-4-(4-nitro-phenyl)-pyrrolidine-3-carboxylic acid, trans-4-(1-naphthyl)-pyrrolidine-3-carboxylic acid, trans-4-(2-naphthyl)-pyrrolidine-3-carboxylic acid, trans-4-(2,5-dichloro-phenyl)-pyrrolidine-3-carboxylic acid, trans-4-(2,3-dichloro-phenyl)-pyrrolidine-3-carboxylic acid, trans-4-(2-trifluoromethyl-phenyl)-pyrrolidine-3-carboxylic acid, trans-4-(3-trifluoromethyl-phenyl)-pyrrolidine-3-carboxylic acid, trans-4-(4-trifluoromethyl-phenyl)-pyrrolidine-3-carboxylic acid, trans-4-(2-cyano-phenyl)-pyrrolidine-3-carboxylic acid, trans-4-(3-cyano-phenyl)-pyrrolidine-3-carboxylic acid, trans-4-(4-cyano-phenyl)-pyrrolidine-3-pyridinylmethyl)-proline, pyridinylmethyl)-proline, trans-4-(2-methoxy-phenyl)-pyrrolidine-3-carboxylic acid, trans-4-(3-methoxy-phenyl)-pyrrolidine-3-carboxylic acid, trans-4-(4-methoxy-phenyl)-pyrrolidine-3-carboxylic acid, trans-4-(2-hydroxy-phenyl)-pyrrolidine-3-carboxylic acid, trans-4-(3-hydroxy-phenyl)-pyrrolidine-3-carboxylic acid, trans-4-(4-hydroxy-phenyl)-pyrrolidine-3-carboxylic acid, trans-4-(2,3-dimethoxy-phenyl)-pyrrolidine-3-carboxylic acid, trans-4-(3,4-dimethoxy-phenyl)-pyrrolidine-3-carboxylic acid, trans-4-(3,5-dimethoxy-phenyl)-pyrrolidine-3-carboxylic acid, trans-4-(2-pyridinyl)-pyrrolidine-3-carboxylic acid, trans-4-(3-pyridinyl)-pyrrolidine-3-carboxylic acid, trans-4-(6-methoxy-3-pyridinyl)-pyrrolidine-3-carboxylic acid, trans-4-(4-pyridinyl)-pyrrolidine-3-carboxylic acid, trans-4-(2-thienyl)-pyrrolidine-3-carboxylic acid, trans-4-(3-thienyl)-pyrrolidine-3-carboxylic acid, trans-4-(2-furanyl)-pyrrolidine-3-carboxylic acid, trans-4-isopropyl-pyrrolidine-3-carboxylic acid, 4-phosphonomethyl-phenylalanine, benzyl-phosphothreonine, (1′-amino-2-phenyl-ethyl)oxirane, (1′-amino-2-cyclohexyl-ethyl)oxirane, (1′-amino-2-[3-bromo-phenyl]ethyl)oxirane, (1′-amino-2-[4-(benzyloxy)phenyl]ethyl)oxirane, (1′-amino-2-[3,5-difluoro-phenyl]ethyl)oxirane, (1′-amino-2-[4-carbamoyl-phenyl]ethyl)oxirane, (1′-amino-2-[benzyloxy-ethyl])oxirane, (1′-amino-2-[4-nitro-phenyl]ethyl)oxirane, (1′-amino-3-phenyl-propyl)oxirane, (1′-amino-3-phenyl-propyl)oxirane, and/or salts and/or protecting group variants thereof.

The term “peptide derivative,” as used herein, refers to a peptide comprising additional chemical or biochemical moieties not normally a part of a naturally occurring peptide. Peptide derivatives include peptides in which the amino-terminus and/or the carboxy-terminus and/or one or more amino acid side chain has been derivatised with a suitable chemical substituent group, as well as cyclic peptides, dual peptides, multimers of the peptides, peptides fused to other proteins or carriers, glycosylated peptides, phosphorylated peptides, peptides conjugated to lipophilic moieties (for example, caproyl, lauryl, stearoyl moieties) and peptides conjugated to an antibody or other biological ligand. Examples of chemical substituent groups that may be used to derivatise a peptide include, but are not limited to, alkyl, cycloalkyl and aryl groups; acyl groups, including alkanoyl and aroyl groups; esters; amides; halogens; hydroxyls; carbamyls, and the like. The substituent group may also be a blocking group such as Fmoc (fluorenylmethyl-O—CO—), carbobenzoxy (benzyl-O—CO—), monomethoxysuccinyl, naphthyl-NH—CO—, acetylamino-caproyl and adamantyl-NH—CO—. Other derivatives include C-terminal hydroxymethyl derivatives, O-modified derivatives (for example, C-terminal hydroxymethyl benzyl ether) and N-terminally modified derivatives including substituted amides such as alkylamides and hydrazides. The substituent group may be a “protecting group” as detailed herein.

The term “peptidomimetic,” as used herein, refers to a compound that is structurally similar to a peptide and contains chemical moieties that mimic the function of the peptide. For example, if a peptide contains two charged chemical moieties having functional activity, a mimetic places two charged chemical moieties in a spatial orientation and constrained structure so that the charged chemical function is maintained in three-dimensional space. The term peptidomimetic thus is intended to include isosteres. The term “isostere,” as used herein, refers to a chemical structure that can be substituted for a peptide because the steric conformation of the chemical structure is similar, for example, the structure fits a binding site specific for the peptide. Examples of peptidomimetics include peptides comprising one or more backbone modifications (i.e., amide bond mimetics), which are well known in the art. Examples of amide bond mimetics include, but are not limited to, —CH₂NH—, —CH₂S—, —CH₂CH₂—, —CHH═CH— (cis and trans), —COCH₂—, —CH(OH)CH₂—, —CH₂SO—, —CS—NH— and —NH—CO— (i.e., a reversed peptide bond) (see, for example, Spatola, Vega Data Vol. 1, Issue 3, (1983); Spatola, in Chemistry and Biochemistry of Amino Acids Peptides and Proteins, Weinstein, ed., Marcel Dekker, New York, p. 267 (1983); Morley, J. S., Trends Pharm. Sci. pp. 463-468 (1980); Hudson et al., Int. J. Pept. Prot. Res. 14:177-185 (1979); Spatola et al., Life Sci. 38:1243-1249 (1986); Hann, J; Chem. Soc. Perkin Trans. 1, 307-314 (1982); Almquist et al., J. Med Chem. 23:1392-1398 (1980); Jennings-White et al., Tetrahedron Lett. 23:2533 (1982); Szelke et al., EP 45665 (1982); Holladay et al., Tetrahedron Lett. 4401-4404(1983); and Hruby, Life Sci. 31:189-199 (1982)). Other examples of peptidomimetics include peptides substituted with one or more benzodiazepine molecules (see, for example, James, G. L. et al. (1993) Science 260:1937-1942) and peptides comprising backbones cross-linked to form lactams or other cyclic structures.

The term “contrast-enhancing” refers to materials capable of being monitored during injection into a mammalian subject by methods for monitoring and detecting such materials, for example by radiography or fluoroscopy. An example of a contrast-enhancing agent is a radiopaque material. Contrast-enhancing agents including radiopaque materials may be either water soluble or water insoluble. Examples of water soluble radiopaque materials include metrizamide, iopamidol, iothalamate sodium, iodomide sodium, and meglumine. Examples of water insoluble radiopaque materials include metals and metal oxides such as gold, titanium, silver, stainless steel, oxides thereof, aluminum oxide, zirconium oxide, etc.

The term “hydrogels,” as used herein, refers to a network of polymer chains that are water-soluble, sometimes found as a colloidal gel in which water is the dispersion medium. In other words, hydrogels are two- or multi-component systems consisting of a three-dimensional network of polymer chains and water that fills the space between the macromolecules, such that the majority of their mass (typically greater than about 80%) is contributed by the entrapped water. Hydrogels are composed of superabsorbent natural or synthetic polymers.

As used herein, a “carbohydrate” (or, equivalently, a “sugar”) is a saccharide (including monosaccharides, oligosaccharides and polysaccharides) and/or a molecule (including oligomers or polymers) derived from one or more monosaccharides, e.g., by reduction of carbonyl groups, by oxidation of one or more terminal groups to carboxylic acids, by replacement of one or more hydroxy group(s) by a hydrogen atom, an amino group, a thiol group or similar heteroatomic groups, etc. The term “carbohydrate” also includes derivatives of these compounds. Non-limiting examples of carbohydrates include allose (“All”), altrose (“Alt”), arabinose (“Ara”), erythrose, erythrulose, fructose (“Fru”), fucosamine (“FucN”), fucose (“Fuc”), galactosamine (“GalN”), galactose (“Gal”), glucosamine (“GlcN”), glucosaminitol (“GlcN-ol”), glucose (“Glc”), glyceraldehyde, 2,3-dihydroxypropanal, glycerol (“Gro”), propane-1,2,3-triol, glycerone (”1,3-dihydroxyacetone”), 1,3-dihydroxypropanone, gulose (“Gul”), idose (“Ido”), lyxose (“Lyx”), mannosamine (“ManN”), mannose (“Man”), psicose (“Psi”), quinovose (“Qui”), quinovosamine, rhamnitol (“Rha-ol”), rhamnosamine (“RhaN”), rhamnose (“Rha”), ribose (“Rib”), ribulose (“Rul”), sialic acid (“Sia” or “Neu”), sorbose (“Sor”), tagatose (“Tag”), talose (“Tal”), tartaric acid, erythraric/threaric acid, threose, xylose (“Xyl”), or xylulose (“Xul”). In some cases, the carbohydrate may be a pentose (i.e., having 5 carbons) or a hexose (i.e., having 6 carbons); and in certain instances, the carbohydrate may be an oligosaccharide comprising pentose and/or hexose units, e.g., including those described above.

A “monosaccharide,” is a carbohydrate or carbohydrate derivative that includes one saccharide unit. Similarly, a “disaccharide,” a “trisaccharide,” a “tetrasaccharide,” a “pentasaccharide,” etc. respectively has 2, 3, 4, 5, etc. saccharide units. A “polysaccharide,” as used herein has multiple saccharide units. In some cases, the carbohydrate is mulitmeric, i.e., comprising more than one saccharide chain.

As used herein, “alginic acid” is a naturally occurring hydrophilic colloidal polysaccharide obtained from the various species of brown seaweed (Phaeophyceae). It occurs in white to yellowish brown filamentous, grainy, granular or powdered forms. It is a linear copolymer consisting mainly of residues of β-1,4-linked D-mannuronic acid and α-1,4-linked L-glucuronic acid. These monomers are often arranged in homopolymeric blocks separated by regions approximating an alternating sequence of the two acid monomers. The formula weight of the structural unit is 176.13 (theoretical; 200 is the actual average). The formula weight of the macromolecule ranges from about 10,000 to about 600,000 (typical average). “Sodium alginate” and “potassium alginate” are salts of alginic acid.

As used herein, “gellan gum” is a high molecular weight polysaccharide gum produced by a pure culture fermentation of a carbohydrate by Pseudomonas elodea, purified by recovery with isopropyl alcohol, dried, and milled. The high molecular weight polysaccharide is principally composed of a tetrasaccharide repeating unit of one rhamnose, one glucuronic acid, and two glucose units, and is substituted with acyl (glyceryl and acetyl) groups as the 0-glycosidically-linked esters. The glucuronic acid is neutralized to a mixed potassium, sodium, calcium, and magnesium salt. It usually contains a small amount of nitrogen containing compounds resulting from the fermentation procedures. It has a formula weight of about 500,000. “Sodium gellan” and “potassium gellan” are salts of gellan gum.

As used herein, “poly vinyl alcohol” (PVA) is a water soluble polymer synthesized by hydrolysis of a poly vinyl ester such as the acetate and used for preparation of fibers. PVA is a thermoplastic that is produced from full or partial hydrolysis of vinyl ester such as vinyl acetate resulting in the replacement of some or all of the acetyl groups with hydroxyl groups. For example: —[CH₂CH(OH)]ₙCH₂CH(COOCH₃)—, where n is zero or a positive integer. In certain embodiments polyvinyl alcohol (PVA) is a synthetic resin produced by polymerisation of vinyl acetate (VAM) followed by hydrolysis of the polyvinyl acetate (PVAc) polymer. The degree of polymerisation determines the molecular weight and viscosity in solution. The degree of hydrolysis (saponification) signifies the extent of conversion of the polyvinyl acetate to the polyvinyl alcohol For example n (degree of hydrolysis) may be in the range of about 68.2 to about 99.8 mol. % and the MW (weight average molecular weight) may range from about 10,000 to about 190,000.

As used herein, “hyaluronic acid” (HA) is a polymer composed of repeating dimeric units of glucuronic acid and N-acetyl glucosamine. It may be of extremely high molecular weight (up to several million daltons) and forms the core of complex proteoglycan aggregates found in extracellular matrix. HA is comprised of linear, unbranching, polyanionic disaccharide units consisting of glucuronic acid (GlcUA) an N-acetyl glucosamine (GlcNAc) joined alternately by β-1-3 and β-1-4 glycosidic bonds. It is a member of the glycosaminoglycan family which includes chondroitin sulphate, dermatin sulphate and heparan sulphate. Unlike other members of this family, it is not found covalently bound to proteins. When incorporated into a neutral aqueous solution hydrogen bond formation occurs between water molecules and adjacent carboxyl and N-acetyl groups. This imparts a conformational stiffniess to the polymer, which limits its flexibility. The hydrogen bond formation results in the unique water-binding and retention capacity of the polymer. It also follows that the water-binding capacity is directly related to the molecular weight of the molecule. Up to six liters of water may be bound per gram of HA. HA solutions are characteristically viscoelastic and pseudoplastic. This rheology is found even in very dilute solutions of the polymer where very viscous gels are formed. The viscoelastic property of HA solutions which is important in its use as a biomaterial is controlled by the concentration and molecular weight of the HA chains. The molecular weight of HA from different sources is polydisperse and highly variable ranging from 10⁴ to 10⁷ Da. The extrusion of HA through the cell membrane as it is produced permits unconstrained polymer elongation and hence a very high molecular weight molecule.

As used herein, “chondroitin sulfate” (CS) refers to unbranched polysaccharides of variable length containing two alternating monosaccharides: D-glucuronic acid (GlcA) and N-acetyl-D-galactosamine (GalNac). Some GlcA residues are epimerized into L-iduronic acid (IdoA); the resulting disaccharide is then referred to as dermatan sulfate. Each monosaccharide may be left unsulfated, sulfated once, or sulfated twice. Most commonly the hydroxyls of the 4 and 6 positions of the N-acetyl-galactosamine are sulfated. Sulfation is mediated by specific sulfotransferases.

As used herein, “heparan sulfate” refers to a member of the glycosaminoglycan family of carbohydrates and is very closely related in structure to heparin. Both consist of a variably sulfated repeating disaccharide unit. The main disacchride units that occurs in heparan sulfate and heparin are GlcA-GlcNAc, GlcA-GlcNS, IdoA-GIcNS, IdoA(2S)-GlcNS, IdoA-GlcNS(6S), and IdoA(2S)-GlcNS(6S); wherein GIcA is β-L-glucuronic acid, IdoA is α-1-iduronic acid, IdoA(2S) is 2-O-sulfo-α-1-iduronic acid, GlcNAc is 2-deoxy-2-acetamido-α-D-glucopyranosyl, GlcNS is 2-deoxy-2-sulfamido-α-D-glucopyranosyl, and GlcNS(6S) is 2-deoxy-2-sulfamido-α-D-glucopyranosyl-6-O-sulfate.

As used herein, “pentosan sulfate” is a sulfated chain of linked xylose sugars.

As used herein, “keratan sulfate,” also called keratosulfate, is any of several sulfated glycosaminoglycans that have been found especially in the cornea, cartilage, and bone.

As used herein, “mucopolysaccharide polysulfate” is polymerized 2-acetamido-2-deoxy-D-galacto-D-glucuronoglycan polysulfate.

As used herein, “carrageenan” consists of alternating 3-linked-β-D-galactopyranose and 4-linked-α-D-galactopyranose units. Carrageenans are linear polymers of about 25,000 galactose derivatives with regular but imprecise structures, dependent on the source and extraction conditions. Idealized structures are described below; -carrageenan, for example, has been found to contain a small proportion of the dimer associated with ι-carrageenan.

κ-Carrageenan (kappa-carrageenan) is -(1→3)-β-D-galactopyranose-4-sulfate-(1→4)-3,6-anhydro-α-D-galactopyranose-(1→3)-. κ-carrageenan is produced by alkaline elimination from μ-carrageenan isolated mostly from the tropical seaweed Kappaphycus alvarezii (also known as Eucheuma cottonii). The experimental charge/dimer is 1.03 rather than 1.0 with 0.82 molecules of anhydrogalactose rather than one.

ι-Carrageenan (iota-carrageenan) is -(1→3)-β-D-galactopyranose-4-sulfate-(1→4)-3,6-anhydro-α-D-galactopyranose-2-sulfate-1→3)-. ι-carrageenan is produced by alkaline elimination from ν-carrageenan isolated mostly from the Philippines seaweed Eucheuma denticulatum (also called Spinosum). The experimental charge/dimer is 1.49 rather than 2.0 with 0.59 molecules of anhydrogalactose rather than one. The three-dimensional structure of the ι-carrageenan double helix has been determined as forming a half-staggered, parallel, threefold, right-handed double helix, stabilized by interchain O2-H—O-5 and O6-H—O-2 hydrogen bonds between the β-D-galactopyranose-4-sulfate units.

λ-Carrageenan (lambda-carrageenan) is -(1→3)-β-D-galactopyranose-2-sulfate-(1→4)-α-D-galactopyranose-2,6-disulfate-(1→3). λ-carrageenan (isolated mainly from Gigartina pistillata or Chondrus crispus) is converted into θ-carrageenan (theta-carrageenan) by alkaline elimination, but at a much slower rate than causes the production of ι-carrageenan and κ-carrageenan. The experimental charge/dimer is 2.09 rather than 3.0 with 0.16 molecules of anhydrogalactose rather than zero.

All carrageenans are highly flexible molecules which, at higher concentrations, wind around each other to form double-helical zones. Gel formation in κ- and ι-carrageenans involves helix formation on cooling from a hot solution together with gel-inducing and gel-strengthening K⁺ or Ca²⁺cations respectively (not Na⁺, although Na⁺ does take part in an aggregation process to form weak gels with κ-carrageenan due to phase separation), which not only aid helix formation but subsequently support aggregating linkages between the helices so forming the junction zones. The strongest gels of κ-carrageenan are formed with K⁺ rather than Li+, Na⁺, Mg²⁺, Ca²⁺, or Sr²⁺. Incomplete formation of ¹C₄ 3,6-anhydro-links will reduce the extent of helix formation as the unbridged α-linked galactose residues may flip to the ⁴C₁ conformation.

The phrase “polydispersity index” refers to the ratio of the “weight average molecular weight” to the “number average molecular weight” for a particular polymer; it reflects the distribution of individual molecular weights in a polymer sample.

The phrase “weight average molecular weight” refers to a particular measure of the molecular weight of a polymer. The weight average molecular weight is calculated as follows: determine the molecular weight of a number of polymer molecules; add the squares of these weights; and then divide by the total weight of the molecules.

The phrase “number average molecular weight” refers to a particular measure of the molecular weight of a polymer. The number average molecular weight is the common average of the molecular weights of the individual polymer molecules. It is determined by measuring the molecular weight of n polymer molecules, summing the weights, and dividing by n.

Polycationic Sclerosing Agents

In certain embodiments, the present invention makes use of compounds which damage lung tissue. For example, in some embodiments a sclerosing agent can be used as part of the administered composition. In some embodiments, the sclerosing agent may be administered alone; or it may be administered separately at the same time as, before, or after other components of the present invention. The sclerosing agent can serve to bring about scar tissue formation, and/or fibroblast proliferation, and/or collagen synthesis, facilitating sealing of regions of damaged lung tissue. In certain embodiments the sclerosing agents that may be used in the present invention are polycations. When a polycation is used, a polyanion may also be used; cytotoxicity of the polycation can be “tuned” by changing the amount of polycation and amount of polyanion used. Polyelectrolytes of the invention are discussed in more detail below.

Polyelectrolytes are polymers whose repeat units bear an electrolyte group. These groups can dissociate in aqueous solutions (e.g., water), making some or all of the polymer repeat units charged. After such electrolytic dissociation, the polymeric species is called a polycation or polyanion, if its repeat units are positively or negatively charged, respectively. A polyelectrolyte that gives rise to a polymer bearing both positive and negative charges after electrolytic dissociation is called an amphoteric polyelectrolyte, or polyampholyte. The generic term “polyion” or “polyionic” is used to refer to electrolytically dissociated polymers of unspecified charge. The ions that dissociate from the polymer are known as counterions.

Polyions can be further divided into “weak” and “strong” types. A “strong” polyion is one which completely retains its charge in solution for most reasonable pH values. A “weak” polyion is one whose charge can be substantially changed by proton transfer to or from the aqueous medium, in the pH range of about 2 to about 10. Thus, weak polyions may not be fully charged in solution and their fractional charge can be modified by changing the solution pH.

Polycations can be any of a variety of compounds having multiple positive charges and a net positive charge. In certain embodiments of the invention the polycations may fall under the class of synthetic polypeptides, also known as polyamino acids. A synthetic polypeptide may be a homopolymer of one of the positively charged (i.e., basic) amino acids such as lysine, arginine, or histidine, or a heteropolymer of two or more positively charged amino acids. In some embodiments, the polycation may be poly-D-lysine, poly-L-lysine, poly-DL-lysine, polyarginine, and polyhistidine. In addition, the polymer may comprise one or more positively charged non-standard amino acids such as omithine, 5-hydroxylysine and the like. Or, the polypeptide may be functionalized with other groups, such as poly(γ-benzyl-L-glutamate). Any or a combination amino acids can be polymerized into linear, branched, or cross-linked chains to generate polycationic polypeptides which are useful components in the compositions and methods described herein. Such polycationic polypeptides may contain at least 100 amino acid residues, at least 200 amino acid residues, at least 300 amino acid residues, at least 500 amino acid residues, at least 750 amino acids, at least 1000 amino acids, at least 2000 amino acids, at least 3000 amino acids, at least 4000 amino acids or more (e.g., from about 20 to about 150 amino acid residues, from about 50 to about 150 amino acid residues, or from about 50 to about 100 amino acid residues). Synthetic polypeptides can be produced by methods known to those of ordinary skill in the art, for example, by chemical synthetic methods or recombinant methods.

The polycationic polymers of the invention may have different degrees of interconnection between repeat units. In one embodiment, a polycationic polymer is a linear polymer, a polymer composed of a single continuous chain of repeat units. In another embodiment, a polycation polymer is a branched polymer, a polymer that includes side chains of repeat units connecting onto the main chain of repeat units (which may be different from side chains already present in the monomers). In another embodiment, a polycation polymer is a crosslinked polymer, a polymer that includes interconnections between chains, either formed during polymerization (i.e., by choice of monomer) or after polymerization (i.e., by adding a specific reagent). In yet another embodiment, a polycation polymer is a network polymer, a crosslinked polymer that includes numerous interconnections between chains such that the entire polymer is, or could be, a single molecule.

Polycationic compositions may be substantially monodisperse or substantially polydisperse. A substantially monodisperse composition comprises polymer molecules, substantially all of which have the same chain length. A substantially polydisperse composition comprises polymer molecules with a variety of chain lengths (and hence molecular weights).

Polycations can have a wide range of molecular weights. The molecular weight of a polycation in a polycationic composition can vary depending on the particular polycationic compound (e.g., a polypeptide), the rate of release of the polycation (e.g., from a gel), the degree of potency desired, etc. In some embodiments, a polycation can have a molecular weight greater than about 10 kD, greater than about 15 kD, greater than about 20 kD, greater than about 25 kD, greater than about 30 kD, greater than about 40 kD, greater than about 50 kD, or greater than about 60 kD, greater than about 70 kD, greater than about 80 kD, greater than about 90 kD, greater than about 100 kD, greater than about 150 kD, greater than about 200 kD, or greater. In other embodiments, a polycation can have a molecular weight between 10-500 kD, between, between 10-250 kD, or between 10-200 kD. However, other sizes may be used as the invention is not limited in this respect. Molecular weights can be determined by those of ordinary skill in the art by methods such as size-exclusion chromatography and/or multi-angle laser light scattering techniques.

The relative basicity of a polycation can vary. In some cases, a polycationic composition comprises a “strong” polycation, which completely retains its charge in solution for most reasonable pH values. In other cases, a polycationic composition comprises a ‘weak’ polycation, i.e., whose charge can be substantially changed by proton transfer to or from the aqueous medium, in the pH range of about 2 to about 10. Polycations of different basicity can be used in polycationic compositions of the invention. A polycation may have a pKb value, for instance, between 2-10, between 6-10, or between 8-10.

Polycations can have varying degrees of solubility in a composition (e.g., varying degrees of water solubility) and/or when delivered to a target region. The solubility of a polycation can be changed, for example, by complexing the polycation with a polyanion, by solvent changes (e.g., by changing the ionic strength of the solvent), and by temperature changes. Polycations can be present in a polycationic composition as a solid (e.g., a precipitate), a gel, or a solution.

If desired, polycations can be combined with an appropriate amount of an agent in a polycationic composition. Agents may be pharmacologically active, meaning they may induce a desired systemic or local effect in addition to the effect of the polycation, or agents may be pharmacologically inactive. In one embodiment, the agent complexes with the polycation in the polycationic composition. In another embodiment, the agent may act as a carrier agent for the polycation or another component of the composition. In another embodiment, the agent may control the release of the polycation from the polycationic composition into the target region. In another embodiment, the agent can modulate (e.g., increase or decrease) the potency of the polycation or another component of the composition. In some cases, the agent may have one or more of the functions listed above, or, the agent may be added to the composition for different purposes.

In some cases, the agent is a polyanion. Any of a variety of polyanions may be used, non-limiting examples including glycosaminoglycans, such as chondroitin sulfate, heparin/heparan sulfate, keratin sulfate, dermatan sulfate, and hyaluronic acid, synthetic polypeptides such as polyglutamic acid and polyaspartic acid, and randomly-structured nucleic acids. Of course, the amount, molecular weight, degree of branching, etc. of the agent in the composition can vary.

According to certain embodiments of the invention, polycations can be complexed with agents such as polyanions. Polycations and polyanions can be weakly or strongly complexed. In some instances, the rate of delivery of a polycation to a targeted area, and/or the potency of a polycation, can be controlled by complexing the polycation with a suitable polyanion. For example, polylysine can be complexed with chondroitin sulfate (CS) and the toxicity of polylysine in a composition can be decreased by adding appropriate amounts of CS. In preferred embodiments, a polyanion is added in an amount sufficient to counterbalance some (e.g, 30%, 40%, 50%, 60%, 70%, 80%, 90%, etc.), but not all, of the positive charges on the polycation. It should be appreciated that the number of positive charges on a polycation and the number of negative charges on a polyanion can be determined and the amount of each molecule to be added can be calculated such that the resulting complex retains a net positive charge. For example, adding equal weights of polylysine and chondroitin sulfate results in a complex with a net positive charge (based on the molecular weights of lysine and chondroitin sulfate and based on a charge of +1 per lysine moiety and −2 per chondroitin sulfate moiety). In some embodiments, polylysirie molecules of approximately 100 kD size are used. The size of the polycation that is used will determine, in part, the net charge per molecule of polycation that is retained after complexation with a predetermined amount of counterion.

In some cases, polycations and polyanions can be complexed into nanoparticles. In one embodiment, a polycation and a polyanion are complexed into micelles, whose sizes can be modified by changing the chain lengths of the polymer. In another embodiment, polycations and polyanions can form polyelectrolyte multilayers (PEMs). PEMs are multilayer complexes comprising alternating layers of polycations and polycations. One or more of the layers may be, or may include, a therapeutically active compound that can be delivered to a targeted area of a patient.

In another embodiment, a polycationic composition comprises a polycation having a number of its positive charges neutralized while the polycation has an overall net positive charge. For instance, the average polycation of the composition may have 10-15%, 15-20%, 20-25%, 25-30%, 30-40%, 40-50%, 50-55%, 55-60%, 60-65%, 65-70%, 70-75%, 75-80%, 80-85%, 85-90%, 90-95%, or 95-99% of its positive charges neutralized.

In aspects of the invention, polycationic compositions can be provided in a number of different forms for administration. For instance, a polycationic composition may be in the form of a solid, solution, suspension, foam, or a gel.

In certain aspects, a polycationic composition may be provided in a form that can be localized when administered to a subject (e.g., substantially restricted to a region of administration in the body of the subject). However, it should be appreciated that in some embodiments a polycationic composition of the invention may be provided and administered as a solution or solid (e.g., powder) without any carrier compound or matrix material (e.g., without a gel or cream etc.).

Accordingly, aspects of the invention involve methods and compositions for localizing polycations within certain regions of the body. In some instances, localization can prevent leakage of harmful amounts of polycations into the circulation where the polycation may be toxic. Localization may also limit the effects of polycations (e.g., sclerosis and fibrosis) to the specific site of administration. In one particular aspect, localization can be achieved by administering a polycationic composition comprising a gel. In another aspect, localization can be achieved by combining a polycation with a second species, such as a polyanion.

In certain embodiments, biodisintegrable polyelectrolytes (polycations and polyanions) can be used. As used herein, a “biodisintegrable material” is a material that undergoes dissolution, degradation, absorption, erosion, corrosion, resorption and/or other disintegration processes in a patient. For instance, the polyelectrolytes can degrade under physiological conditions in between about 1 to about 12 weeks; about 1 to about 6 weeks; about 1 to about 4 weeks; about 2 to about 10 weeks; about 2 to about 5 weeks; or about 2 to about 4 weeks.

Forms for Administration

In aspects of the invention, polycationic compositions can be provided in a number of different forms for administration. For instance, a polycationic composition may be in the form of a solid, solution, suspension, foam, or a gel.

In certain aspects, a polycationic composition may be provided in a form that can be localized when administered to a subject (e.g., substantially restricted to a region of administration in the body of the subject). However, it should be appreciated that in some embodiments a polycationic composition of the invention may be provided and administered as a solution or solid (e.g., powder) without any carrier compound or matrix material (e.g., without a gel or cream etc.).

In some embodiments, polycation compositions are provided in association with a gel. A polycation may be soluble within the gel matrix. In some embodiments, a polycation may interact with one or more components of the gel matrix. The gel may be biocompatible, and can be designed with selected properties of compliancy and elasticity for different therapeutic applications. In some cases, the gel is also biodegradable.

A variety of different gels can be used in accordance with the present invention, including, but not limited to: hydrogels, alginate, acrylamide, agarose, mixtures thereof, and the like. Gels may comprise biological, biochemical, and/or synthetic components or a combination thereof. For example gels may be protein-based gels such as fibrin, collagen, keratin, gelatin; carbohydrate derived gels such as starch, chitin, chitosan, carboxymethylcellulose or cellulose, and/or their biologically compatible derivatives.

In one embodiment, the gel may rapidly polymerize at the specific site of administration. The rate of polymerization of the gel can be controlled by varying the chemical makeup of the gel (e.g., degree of branching), molecular weight. Gels can be polymerized chemically, or by light, heat, exposure to oxygen (e.g., air), or other methods. In certain embodiments, a gel may form a firm mechanical solid upon polymerization.

It should be appreciated that one or more alternative forms of administration also may be used (e.g., creams, colloidal preparations, viscous preparations, etc.).

In another aspect, the invention provides methods for ensuring that the effects of one or more polycations are essentially limited to a specific site of administration by complexing them with one or more polyanions to prevent leakage of the material into the circulation where the polycation(s) may be toxic. In certain embodiments, a polycation-polyanion complex may be incorporated into an injectable system (e.g., an injectible hydrogel system) that can be delivered to and maintained at specific site (e.g., by rapidly polymerizing the hydrogel). The hydrogel may be a biological hydrogel or synthetic hydrogel.

In certain embodiments, hydrogels suitable for use in the invention crosslink upon the addition of the crosslinker, i.e., without the need for a separate energy source. Such systems allow good control of the crosslinking process, because gelation does not occur until the mixing of the two solutions takes place. If desired, polymer solutions may contain dyes or other means for visualizing the hydrogel. The crosslinkable solutions also may contain a bioactive drug or therapeutic compound that is entrapped in the resulting hydrogel, so that the hydrogel becomes a drug delivery vehicle.

Properties of the hydrogel system, other than crosslinkability, preferably should be selected on the basis of exhibited biocompatibility and lack of toxicity. Additionally, the hydrogel precursor solutions should not contain harmful or toxic solvents. Preferably, the hydrogel precursors are substantially soluble in water to allow application in a physiologically compatible solution, such as buffered isotonic saline. It is also preferable that the hydrogel be biodegradable, so that it does not have to be retrieved from the body. Biodegradability, as used herein, refers to the predictable disintegration of the hydrogel into molecules small enough to be metabolized or excreted under normal physiological conditions.

Selected Compositions of the Invention

One aspect of the present invention relates to a composition comprising a polycation and a polyanion; wherein the ratio of X to Y is greater than about one; X is the product of the mass of the polycation and the charge-per-mass ratio of the polycation; and Y is the product of the mass of the polyanion and the change-per-mass ratio of the polyanion.

In certain embodiments, the present invention relates to the aforementioned composition, wherein said composition consists essentially of the polycation and the polyanion.

In certain embodiments, the present invention relates to the aforementioned composition, wherein said composition consists of the polycation and the polyanion.

In certain embodiments, the present invention relates to the aforementioned composition, wherein said composition is a solid at ambient temperature or physiological temperature.

In certain embodiments, the present invention relates to the aforementioned composition, further comprising fibrin, fibrinogen, polyvinyl alcohol, alginate or gellan.

In certain embodiments, the present invention relates to the aforementioned composition,, further comprising fibrinogen.

In certain embodiments, the present invention relates to the aforementioned composition, further comprising thrombin, borate, boronate, calcium, or magnesium.

In certain embodiments, the present invention relates to the aforementioned composition, further comprising thrombin.

In certain embodiments, the present invention relates to the aforementioned composition, further comprising calcium chloride.

In certain embodiments, the present invention relates to the aforementioned composition, further comprising a hydrogel formed from the combination of fibrin and thrombin; fibrinogen and thrombin; polyvinyl alcohol and borate; polyvinyl alcohol and a boronate; alginate and calcium; or gellan and magnesium.

In certain embodiments, the present invention relates to the aforementioned composition, further comprising a hydrogel formed from the combination of fibrinogen and thrombin.

In certain embodiments, the present invention relates to the aforementioned composition, wherein said polycation has a molecular weight greater than about 10 kD and less than about 500 kD.

In certain embodiments, the present invention relates to the aforementioned composition, wherein said polycation has a molecular weight greater than about 10 kD and less than about 250 kD.

In certain embodiments, the present invention relates to the aforementioned composition, wherein said polycation has a molecular weight greater than about 10 kD and less than about 200 kD.

In certain embodiments, the present invention relates to the aforementioned composition, wherein said polycation is a poly(amino acid).

In certain embodiments, the present invention relates to the aforementioned composition, wherein said polycation is a poly(amino acid); and said polycation contains at least about 50 amino acid residues and less than about 4000 amino acid residues.

In certain embodiments, the present invention relates to the aforementioned composition, wherein said polycation is a poly(amino acid); and said polycation contains at least about 100 amino acid residues and less than about 4000 amino acid residues.

In certain embodiments, the present invention relates to the aforementioned composition, wherein said polycation is a poly(amino acid); and said polycation contains at least about 200 amino acid residues and less than about 4000 amino acid residues.

In certain embodiments, the present invention relates to the aforementioned composition, wherein said polycation is a poly(amino acid); and said polycation contains at least about 300 amino acid residues and less than about 4000 amino acid residues.

In certain embodiments, the present invention relates to the aforementioned composition, wherein said polycation is a poly(amino acid); and said polycation contains at least about 500 amino acid residues and less than about 4000 amino acid residues.

In certain embodiments, the present invention relates to the aforementioned composition, wherein said polycation is a poly(amino acid); and said polycation contains at least about 750 amino acid residues and less than about 4000 amino acid residues.

In certain embodiments, the present invention relates to the aforementioned composition, wherein said polycation is a poly(amino acid); and said polycation contains at least about 1000 amino acid residues and less than about 4000 amino acid residues.

In certain embodiments, the present invention relates to the aforementioned composition, wherein said polycation is a poly(amino acid); and said polycation contains at least about 2000 amino acid residues and less than about 4000 amino acid residues.

In certain embodiments, the present invention relates to the aforementioned composition, wherein said polycation is a poly(amino acid); and said polycation contains at least about 3000 amino acid residues and less than about 4000 amino acid residues

In certain embodiments, the present invention relates to the aforementioned composition, wherein said polycation is a poly(amino acid); said poly(amino acid) comprises a plurality of amino acids independently selected from the group consisting of Asp, Glu, Lys, Orn, Arg, Gly, Ala, Val, Leu, Ile, Met, Pro, Phe, Trp, Asn, Gln, Ser, Thr, Tyr, Cys, and His; provided that no less than about twenty-five percent of the amino acids are independently selected from the group consisting of Lys, Om, His and Arg; further provided that no more than five percent of the amino acids are independently selected from the group consisting of Asp and Glu.

In certain embodiments, the present invention relates to the aforementioned composition, wherein said polycation is a poly(amino acid); said poly(amino acid) is represented by poly(X-Y), poly(X-Y-Y), or poly(X-Y-Y-Y); X is independently for each occurrence Lys, Om, His or Arg; and Y is independently for each occurrence Gly, Ala, Val, Leu, Ile, Met, Pro, Phe, Trp, Asn, Gln, Ser, Thr, Tyr, or Cys.

In certain embodiments, the present invention relates to the aforementioned composition, wherein said polycation is a poly(amino acid); said poly(amino acid) is represented by poly(X-Y), poly(X-Y-Y), or poly(X-Y-Y-Y); X is Lys; and Y is independently for each occurrence Gly, Ala, Val, Leu, Ile, Met, Pro, Phe, Trp, Asn, Gln, Ser, Thr, Tyr, Cys, or His.

In certain embodiments, the present invention relates to the aforementioned composition, wherein said polycation is poly(Lys), poly(Orn), poly(Arg) or poly(His).

In certain embodiments, the present invention relates to the aforementioned composition, wherein said polycation is poly(Lys).

In certain embodiments, the present invention relates to the aforementioned composition, wherein said polycation is poly(L-Lys).

In certain embodiments, the present invention relates to the aforementioned composition, wherein said polycation degrades under physiological conditions in about 1 to about 12 weeks.

In certain embodiments, the present invention relates to the aforementioned composition, wherein said polycation degrades under physiological conditions in about 1 to about 6 weeks.

In certain embodiments, the present invention relates to the aforementioned composition, wherein said polycation degrades under physiological conditions in about 1 to about 4 weeks.

In certain embodiments, the present invention relates to the aforementioned composition, wherein said polycation degrades under physiological conditions in about 2 to about 5 weeks.

In certain embodiments, the present invention relates to the aforementioned composition, wherein said polyanion has a molecular weight greater than about 10 kD and less than about 500 kD.

In certain embodiments, the present invention relates to the aforementioned composition, wherein said polyanion has a molecular weight greater than about 20 kD and less than about 250 kD.

In certain embodiments, the present invention relates to the aforementioned composition, wherein said polyanion has a molecular weight greater than about 20 kD and less than about 100 kD.

In certain embodiments, the present invention relates to the aforementioned composition, wherein said polyanion is a poly(saccharide).

In certain embodiments, the present invention relates to the aforementioned composition, wherein said polyanion is a poly(saccharide); and said polyanion contains at least about 5 saccharide residues and less than about 2,500 saccharide residues.

In certain embodiments, the present invention relates to the aforementioned composition, wherein said polyanion is a poly(saccharide); and said polyanion contains at least about 20 saccharide residues and less than about 2,500 saccharide residues.

In certain embodiments, the present invention relates to the aforementioned composition, wherein said polyanion is a poly(saccharide); and said polyanion contains at least about 50 saccharide residues and less than about 2,500 saccharide residues.

In certain embodiments, the present invention relates to the aforementioned composition, wherein said polyanion is a poly(saccharide); and said polyanion contains at least about 100 saccharide residues and less than about 2,500 saccharide residues.

In certain embodiments, the present invention relates to the aforementioned composition, wherein said polyanion is a poly(saccharide); and said polyanion contains at least about 200 saccharide residues and less than about 2,500 saccharide residues.

In certain embodiments, the present invention relates to the aforementioned composition, wherein said polyanion is a poly(saccharide); and said polyanion contains at least about 300 saccharide residues and less than about 2,500 saccharide residues.

In certain embodiments, the present invention relates to the aforementioned composition, wherein said polyanion is a poly(saccharide); and said polyanion contains at least about 500 saccharide residues and less than about 2,500 saccharide residues.

In certain embodiments, the present invention relates to the aforementioned composition, wherein said polyanion is a poly(saccharide); and said polyanion contains at least about 750 saccharide residues and less than about 2,500 saccharide residues.

In certain embodiments, the present invention relates to the aforementioned composition, wherein said polyanion is a poly(saccharide); and said polyanion contains at least about 1,000 saccharide residues and less than about 2,500 saccharide residues.

In certain embodiments, the present invention relates to the aforementioned composition, wherein said polyanion is a poly(saccharide); and said polyanion contains at least about 1,500 saccharide residues and less than about 2,500 saccharide residues.

In certain embodiments, the present invention relates to the aforementioned composition, wherein said polyanion is a poly(saccharide); and said polyanion contains at least about 2,000 saccharide residues and less than about 2,500 saccharide residues.

In certain embodiments, the present invention relates to the aforementioned composition, wherein said polyanion is a poly(saccharide); and said saccharides are selected from the group consisting of cellulose, xylose, N-acetyllactosamine, glucuronic acid, mannuronic acid, and guluronic acid.

In certain embodiments, the present invention relates to the aforementioned composition, wherein said polyanion is a poly(saccharide); and a plurality of said saccharides are sulfated.

The composition of claim 1, wherein said polyanion is a poly(saccharide); and a plurality of said saccharides are carboxymethylated.

The composition of claim 1, wherein said polyanion is a poly(saccharide) selected from the group consisting of heparan sulfate, dermatan sulfate, chondroitin sulfate, pentosan sulfate, keratan sulfate, mucopolysaccharide polysulfate, carrageenan, sodium alginate, potassium alginate, hyaluronic acid, and carboxymethylcellulose.

In certain embodiments, the present invention relates to the aforementioned composition, wherein said polyanion is chondroitin sulfate.

In certain embodiments, the present invention relates to the aforementioned composition, wherein said polyanion is a poly(amino acid).

In certain embodiments, the present invention relates to the aforementioned composition, wherein said polyanion is a poly(amino acid); and said polycation contains at least about 50 amino acid residues and less than about 4000 amino acid residues.

In certain embodiments, the present invention relates to the aforementioned composition, wherein said polyanion is a poly(amino acid); and said polycation contains at least about 100 amino acid residues and less than about 4000 amino acid residues.

In certain embodiments, the present invention relates to the aforementioned composition, wherein said polyanion is a poly(amino acid); and said polycation contains at least about 200 amino acid residues and less than about 4000 amino acid residues.

In certain embodiments, the present invention relates to the aforementioned composition, wherein said polyanion is a poly(amino acid); and said polycation contains at least about 300 amino acid residues and less than about 4000 amino acid residues.

In certain embodiments, the present invention relates to the aforementioned composition, wherein said polyanion is a poly(amino acid); and said polycation contains at least about 500 amino acid residues and less than about 4000 amino acid residues.

In certain embodiments, the present invention relates to the aforementioned composition, wherein said polyanion is a poly(amino acid); and said polycation contains at least about 750 amino acid residues and less than about 4000 amino acid residues.

In certain embodiments, the present invention relates to the aforementioned composition, wherein said polyanion is a poly(amino acid); and said polycation contains at least about 1000 amino acid residues and less than about 4000 amino acid residues.

In certain embodiments, the present invention relates to the aforementioned composition, wherein said polyanion is a poly(amino acid); and said polycation contains at least about 2000 amino acid residues and less than about 4000 amino acid residues.

In certain embodiments, the present invention relates to the aforementioned composition, wherein said polyanion is a poly(amino acid); and said polycation contains at least about 3000 amino acid residues and less than about 4000 amino acid residues

In certain embodiments, the present invention relates to the aforementioned composition, wherein said polyanion is a poly(amino acid); said poly(amino acid) comprises a plurality of amino acids independently selected from the group consisting of Asp, Glu, Lys, Om, Arg, Gly, Ala, Val, Leu, Ile, Met, Pro, Phe, Trp, Asn, Gln, Ser, Thr, Tyr, Cys, and His; provided that no less than about twenty-five percent of the amino acids are independently selected from the group consisting of Asp and Glu; further provided that no more than five percent of the amino acids are independently selected from the group consisting of Lys, Om, and Arg.

In certain embodiments, the present invention relates to the aforementioned composition, wherein said polyanion is a poly(amino acid); said poly(amino acid) is represented by poly(X-Y), poly(X-Y-Y), or poly(X-Y-Y-Y); X is independently for each occurrence Asp or Glu; and Y is independently for each occurrence Gly, Ala, Val, Leu, Ile, Met, Pro, Phe, Trp, Asn, Gln, Ser, Thr, Tyr, Cys, or His.

In certain embodiments, the present invention relates to the aforementioned composition, wherein said polyanion is poly(Glu).

In certain embodiments, the present invention relates to the aforementioned composition, wherein said polyanion is poly(Asp).

In certain embodiments, the present invention relates to the aforementioned composition, wherein said polyanion degrades under physiological conditions in about 1 to about 12 weeks.

In certain embodiments, the present invention relates to the aforementioned composition, wherein said polyanion degrades under physiological conditions in about 1 to about 6 weeks.

In certain embodiments, the present invention relates to the aforementioned composition, wherein said polyanion degrades under physiological conditions in about 1 to about 4 weeks.

In certain embodiments, the present invention relates to the aforementioned composition, wherein said polyanion degrades under physiological conditions in about 2 to about 5 weeks.

The compositions described above can also contain one or more antibiotics to help prevent infection. Alternatively or in addition, antibiotics can be administered via other routes (e.g., they may be administered orally or intramuscularly).

In certain embodiments, the present invention relates to the aforementioned composition, further comprising an anti-infective; wherein said anti-infective is selected from the group consisting of an aminoglycoside, a tetracycline, a sulfonamide, p-aminobenzoic acid, a diaminopyrimidine, a quinolone, a β-lactam, a β-lactamase inhibitor, chloraphenicol, a macrolide, penicillins, cephalosporins, linomycin, clindamycin, spectinomycin, polymyxin B, colistin, vancomycin, bacitracin, isoniazid, rifampin, ethambutol, ethionamide, aminosalicylic acid, cycloserine, capreomycin, a sulfone, clofazimine, thalidomide, a polyene antifungal, flucytosine, imidazole, triazole, griseofulvin, terconazole, butoconazole ciclopirax, ciclopirox olamine, haloprogin, tolnaftate, naftifine, and terbinafine, or a combination thereof.

In certain embodiments, the present invention relates to the aforementioned composition, further comprising an anti-infective; wherein said anti-infective is tetracycline.

In certain embodiments, the present invention relates to the aforementioned composition, further comprising a contrast-enhancing agent.

In certain embodiments, the present invention relates to the aforementioned composition, further comprising a contrast-enhancing agent; wherein said contrast-enhancing agent is selected from the group consisting of radiopaque materials, paramagnetic materials, heavy atoms, transition metals, lanthanides, actinides, dyes, and radionuclide-containing materials.

Selected Methods of the Invention

Certain aspects of the invention involve methods and compositions for localizing polycations within certain regions of the body. In some instances, localization can prevent leakage of harmful amounts of polycations into the circulation where the polycation may be toxic. Localization may also limit the effects of polycations (e.g., sclerosis and fibrosis) to the specific site of administration. In one particular aspect, localization can be achieved by combining a polycation with a second species, such as a polyanion.

The exact duration of exposure may vary depending upon the specific application. Exposure times can vary depending on the form in which the polycationic composition is administered to the body. For polycationic compositions in the form of gels, exposure times may be defined by the degradation of the hydrogel in some cases. Degradation times of the gel can be adjusted by varying, for instance, the cross-linking density of the gel. Accordingly, in some embodiments a polycationic composition of the invention may be provided in a form that remains at a target tissue site for about about 1 day, about 1 week, about 2 weeks, about 1 month, or several months.

One aspect of the invention relates to a method of inducing scarring and fibrosis at a target area in a subject, comprising the step of administering an amount of a composition to a target area in said subject; wherein said composition comprises a polycation and a polyanion; the ratio of X to Y is greater than about 1; X is the product of the mass of the polycation and the charge-per-mass ratio of the polycation; and Y is the product of the mass of the polyanion and the charge-per-mass ratio of the polyanion.

In certain embodiments, the present invention relates to the aforementioned method, wherein said target area is selected from the group consisting of pulmonary tissue and fallopian tubes.

In certain embodiments, the present invention relates to the aforementioned method, wherein said target area comprises pulmonary tissue.

In certain embodiments, the present invention relates to the aforementioned method, wherein said subject is a human.

In certain embodiments, the present invention relates to the aforementioned method, wherein said subject has emphysema.

In certain embodiments, the present invention relates to the aforementioned method, wherein said subject has suffered a traumatic injury of the lung.

In certain embodiments, the present invention relates to the aforementioned method, wherein said composition is administered via a multi-lumen catheter.

In certain embodiments, the present invention relates to the aforementioned method, wherein said composition is administered via a dual-lumen catheter.

In certain embodiments, the present invention relates to the aforementioned method, wherein said amount is between about 5 mL and about 300 mL.

In certain embodiments, the present invention relates to the aforementioned method, wherein said amount is between about 10 mL and about 100 mL.

In certain embodiments, the present invention relates to the aforementioned method, wherein said amount is between about 10 mL and about 50 mL.

In certain embodiments, the present invention relates to the aforementioned method, wherein said composition consists essentially of the polycation and the polyanion.

In certain embodiments, the present invention relates to the aforementioned method, wherein said composition consists of the polycation and the polyanion.

In certain embodiments, the present invention relates to the aforementioned method, wherein said composition is a solid at ambient temperature or physiological temperature.

In certain embodiments, the present invention relates to the aforementioned method, wherein said polycation has a molecular weight greater than about 10 kD and less than about 500 kD.

In certain embodiments, the present invention relates to the aforementioned method, wherein said polycation has a molecular weight greater than about 10 kD and less than about 250 kD.

In certain embodiments, the present invention relates to the aforementioned method, wherein said polycation has a molecular weight greater than about 10 kD and less than about 200 kD.

In certain embodiments, the present invention relates to the aforementioned method, wherein said polycation is a poly(amino acid).

In certain embodiments, the present invention relates to the aforementioned method, wherein said polycation is a poly(amino acid); and said polycation contains at least about 50 amino acid residues and less than about 4000 amino acid residues.

In certain embodiments, the present invention relates to the aforementioned method, wherein said polycation is a poly(amino acid); and said polycation contains at least about 100 amino acid residues and less than about 4000 amino acid residues.

In certain embodiments, the present invention relates to the aforementioned method, wherein said polycation is a poly(amino acid); and said polycation contains at least about 200 amino acid residues and less than about 4000 amino acid residues.

In certain embodiments, the present invention relates to the aforementioned method, wherein said polycation is a poly(amino acid); and said polycation contains at least about 300 amino acid residues and less than about 4000 amino acid residues.

In certain embodiments, the present invention relates to the aforementioned method, wherein said polycation is a poly(amino acid); and said polycation contains at least about 500 amino acid residues and less than about 4000 amino acid residues.

In certain embodiments, the present invention relates to the aforementioned method, wherein said polycation is a poly(amino acid); and said polycation contains at least about 750 amino acid residues and less than about 4000 amino acid residues.

In certain embodiments, the present invention relates to the aforementioned method, wherein said polycation is a poly(amino acid); and said polycation contains at least about 1000 amino acid residues and less than about 4000 amino acid residues.

In certain embodiments, the present invention relates to the aforementioned method, wherein said polycation is a poly(amino acid); and said polycation contains at least about 2000 amino acid residues and less than about 4000 amino acid residues.

In certain embodiments, the present invention relates to the aforementioned method, wherein said polycation is a poly(amino acid); and said polycation contains at least about 3000 amino acid residues and less than about 4000 amino acid residues In certain embodiments, the present invention relates to the aforementioned method, wherein said polycation is a poly(amino acid); said poly(amino acid) comprises a plurality of amino acids independently selected from the group consisting of Asp, Glu, Lys, Orn, Arg, Gly, Ala, Val, Leu, Ile, Met, Pro, Phe, Trp, Asn, Gln, Ser, Thr, Tyr, Cys, and His; provided that no less than about twenty-five percent of the amino acids are independently selected from the group consisting of Lys, Om, His and Arg; further provided that no more than five percent of the amino acids are independently selected from the group consisting of Asp and Glu.

In certain embodiments, the present invention relates to the aforementioned method, wherein said polycation is a poly(amino acid); said poly(amino acid) is represented by poly(X-Y), poly(X-Y-Y), or poly(X-Y-Y-Y); X is independently for each occurrence Lys, Om, His or Arg; and Y is independently for each occurrence Gly, Ala, Val, Leu, Ile, Met, Pro, Phe, Trp, Asn, Gln, Ser, Thr, Tyr, or Cys.

In certain embodiments, the present invention relates to the aforementioned method, wherein said polycation is a poly(amino acid); said poly(amino acid) is represented by poly(X-Y), poly(X-Y-Y), or poly(X-Y-Y-Y); X is Lys; and Y is independently for each occurrence Gly, Ala, Val, Leu, Ile, Met, Pro, Phe, Trp, Asn, Gln, Ser, Thr, Tyr, Cys, or His.

In certain embodiments, the present invention relates to the aforementioned method, wherein said polycation is poly(Lys), poly(Orm), poly(Arg) or poly(His).

In certain embodiments, the present invention relates to the aforementioned method, wherein said polycation is poly(Lys).

In certain embodiments, the present invention relates to the aforementioned method, wherein said polycation is poly(L-Lys).

In certain embodiments, the present invention relates to the aforementioned method, wherein said polycation degrades under physiological conditions in about 1 to about 12 weeks.

In certain embodiments, the present invention relates to the aforementioned method, wherein said polycation degrades under physiological conditions in about 1 to about 6 weeks.

In certain embodiments, the present invention relates to the aforementioned method, wherein said polycation degrades under physiological conditions in about 1 to about 4 weeks.

In certain embodiments, the present invention relates to the aforementioned method, wherein said polycation degrades under physiological conditions in about 2 to about 5 weeks.

In certain embodiments, the present invention relates to the aforementioned method, wherein said polyanion has a molecular weight greater than about 10 kD and less than about 500 kD.

In certain embodiments, the present invention relates to the aforementioned method, wherein said polyanion has a molecular weight greater than about 20 kD and less than about 250 kD.

In certain embodiments, the present invention relates to the aforementioned method, wherein said polyanion has a molecular weight greater than about 20 kD and less than about 100 kD.

In certain embodiments, the present invention relates to the aforementioned method, wherein said polyanion is a poly(saccharide).

In certain embodiments, the present invention relates to the aforementioned method, wherein said polyanion is a poly(saccharide); and said polyanion contains at least about 5 saccharide residues and less than about 2,500 saccharide residues.

In certain embodiments, the present invention relates to the aforementioned method, wherein said polyanion is a poly(saccharide); and said polyanion contains at least about 20 saccharide residues and less than about 2,500 saccharide residues.

In certain embodiments, the present invention relates to the aforementioned method, wherein said polyanion is a poly(saccharide); and said polyanion contains at least about 50 saccharide residues and less than about 2,500 saccharide residues.

In certain embodiments, the present invention relates to the aforementioned method, wherein said polyanion is a poly(saccharide); and said polyanion contains at least about 100 saccharide residues and less than about 2,500 saccharide residues.

In certain embodiments, the present invention relates to the aforementioned method, wherein said polyanion is a poly(saccharide); and said polyanion contains at least about 200 saccharide residues and less than about 2,500 saccharide residues.

In certain embodiments, the present invention relates to the aforementioned method, wherein said polyanion is a poly(saccharide); and said polyanion contains at least about 300 saccharide residues and less than about 2,500 saccharide residues.

In certain embodiments, the present invention relates to the aforementioned method, wherein said polyanion is a poly(saccharide); and said polyanion contains at least about 500 saccharide residues and less than about 2,500 saccharide residues.

In certain embodiments, the present invention relates to the aforementioned method, wherein said polyanion is a poly(saccharide); and said polyanion contains at least about 750 saccharide residues and less than about 2,500 saccharide residues.

In certain embodiments, the present invention relates to the aforementioned method, wherein said polyanion is a poly(saccharide); and said polyanion contains at least about 1,000 saccharide residues and less than about 2,500 saccharide residues.

In certain embodiments, the present invention relates to the aforementioned method, wherein said polyanion is a poly(saccharide); and said polyanion contains at least about 1,500 saccharide residues and less than about 2,500 saccharide residues.

In certain embodiments, the present invention relates to the aforementioned method, wherein said polyanion is a poly(saccharide); and said polyanion contains at least about 2,000 saccharide residues and less than about 2,500 saccharide residues.

In certain embodiments, the present invention relates to the aforementioned method, wherein said polyanion is a poly(saccharide); and said saccharides are selected from the group consisting of cellulose, xylose, N-acetyllactosamine, glucuronic acid, mannuronic acid, and guluronic acid.

In certain embodiments, the present invention relates to the aforementioned method, wherein said polyanion is a poly(saccharide); and a plurality of said saccharides are sulfated.

In certain embodiments, the present invention relates to the aforementioned method, wherein said polyanion is a poly(saccharide); and a plurality of said saccharides are carboxymethylated.

In certain embodiments, the present invention relates to the aforementioned method, wherein said polyanion is a poly(saccharide) selected from the group consisting of heparan sulfate, dermatan sulfate, chondroitin sulfate, pentosan sulfate, keratan sulfate, mucopolysaccharide polysulfate, carrageenan, sodium alginate, potassium alginate, hyaluronic acid, and carboxymethylcellulose.

In certain embodiments, the present invention relates to the aforementioned method, wherein said polyanion is chondroitin sulfate.

In certain embodiments, the present invention relates to the aforementioned method, wherein said polyanion is a poly(amino acid).

In certain embodiments, the present invention relates to the aforementioned method, wherein said polyanion is a poly(amino acid); and said polycation contains at least about 50 amino acid residues and less than about 4000 amino acid residues.

In certain embodiments, the present invention relates to the aforementioned method, wherein said polyanion is a poly(amino acid); and said polycation contains at least about 100 amino acid residues and less than about 4000 amino acid residues.

In certain embodiments, the present invention relates to the aforementioned method, wherein said polyanion is a poly(amino acid); and said polycation contains at least about 200 amino acid residues and less than about 4000 amino acid residues.

In certain embodiments, the present invention relates to the aforementioned method, wherein said polyanion is a poly(amino acid); and said polycation contains at least about 300 amino acid residues and less than about 4000 amino acid residues.

In certain embodiments, the present invention relates to the aforementioned method, wherein said polyanion is a poly(amino acid); and said polycation contains at least about 500 amino acid residues and less than about 4000 amino acid residues.

In certain embodiments, the present invention relates to the aforementioned method, wherein said polyanion is a poly(amino acid); and said polycation contains at least about 750 amino acid residues and less than about 4000 amino acid residues.

In certain embodiments, the present invention relates to the aforementioned method, wherein said polyanion is a poly(amino acid); and said polycation contains at least about 1000 amino acid residues and less than about 4000 amino acid residues.

In certain embodiments, the present invention relates to the aforementioned method, wherein said polyanion is a poly(amino acid); and said polycation contains at least about 2000 amino acid residues and less than about 4000 amino acid residues.

In certain embodiments, the present invention relates to the aforementioned method, wherein said polyanion is a poly(amino acid); and said polycation contains at least about 3000 amino acid residues and less than about 4000 amino acid residues

In certain embodiments, the present invention relates to the aforementioned method, wherein said polyanion is a poly(amino acid); said poly(amino acid) comprises a plurality of amino acids independently selected from the group consisting of Asp, Glu, Lys, Orn, Arg, Gly, Ala, Val, Leu, Ile, Met, Pro, Phe, Trp, Asn, Gln, Ser, Thr, Tyr, Cys, and His; provided that no less than about twenty-five percent of the amino acids are independently selected from the group consisting of Asp and Glu; further provided that no more than five percent of the amino acids are independently selected from the group consisting of Lys, Orn, and Arg.

In certain embodiments, the present invention relates to the aforementioned method, wherein said polyanion is a poly(amino acid); said poly(amino acid) is represented by poly(X-Y), poly(X-Y-Y), or poly(X-Y-Y-Y); X is independently for each occurrence Asp or Glu; and Y is independently for each occurrence Gly, Ala, Val, Leu, Ile, Met, Pro, Phe, Trp, Asn, Gln, Ser, Thr, Tyr, Cys, or His.

In certain embodiments, the present invention relates to the aforementioned method, wherein said polyanion is poly(Glu).

In certain embodiments, the present invention relates to the aforementioned method, wherein said polyanion is poly(Asp).

In certain embodiments, the present invention relates to the aforementioned method, wherein said polyanion degrades under physiological conditions in about 1 to about 12 weeks.

In certain embodiments, the present invention relates to the aforementioned method, wherein said polyanion degrades under physiological conditions in about 1 to about 6 weeks.

In certain embodiments, the present invention relates to the aforementioned method, wherein said polyanion degrades under physiological conditions in about 1 to about 4 weeks.

In certain embodiments, the present invention relates to the aforementioned method, wherein said polyanion degrades under physiological conditions in about 2 to about 5 weeks.

In certain embodiments, the present invention relates to the aforementioned method, wherein said composition further comprises fibrin, fibrionogen, polyvinyl alcohol, alginate or gellan.

In certain embodiments, the present invention relates to the aforementioned method, wherein said composition further comprises fibrinogen.

In certain embodiments, the present invention relates to the aforementioned method, wherein said composition further comprises thrombin, borate, boronate, calcium, or magnesium.

In certain embodiments, the present invention relates to the aforementioned method, wherein said composition further comprises thrombin.

In certain embodiments, the present invention relates to the aforementioned method, wherein said composition further comprises an anti-infective; wherein said anti-infective is selected from the group consisting of an aminoglycoside, a tetracycline, a sulfonamide, p-aminobenzoic acid, a diaminopyrimidine, a quinolone, a β-lactam, a β-lactamase inhibitor, chloraphenicol, a macrolide, penicillins, cephalosporins, linomycin, clindamycin, spectinomycin, polymyxin B, colistin, vancomycin, bacitracin, isoniazid, rifampin, ethambutol, ethionamide, aminosalicylic acid, cycloserine, capreomycin, a sulfone, clofazimine, thalidomide, a polyene antifungal, flucytosine, imidazole, triazole, griseofulvin, terconazole, butoconazole ciclopirax, ciclopirox olamine, haloprogin, tolnaftate, naftifine, and terbinafine, or a combination thereof.

In certain embodiments, the present invention relates to the aforementioned method, wherein said composition further comprises an anti-infective; wherein said anti-infective is tetracycline.

In certain embodiments, the present invention relates to the aforementioned method, wherein said composition further comprises a contrast-enhancing agent.

In certain embodiments, the present invention relates to the aforementioned method, wherein said composition further comprises a contrast-enhancing agent; wherein said contrast-enhancing agent is selected from the group consisting of radiopaque materials, paramagnetic materials, heavy atoms, transition metals, lanthanides, actinides, dyes, and radionuclide-containing materials.

Selected Kits of the Invention

One aspect of the present invention relates to a kit, comprising: a container comprising a composition comprising a polycation and a polyanion; and instructions for use thereof in lung volume reduction therapy; wherein the ratio of X to Y is greater than about 1; X is the product of the mass of the polycation and the charge-per-mass ratio of the polycation; and Y is the product of the mass of the polyanion and the charge-per-mass ratio of the polyanion.

In certain embodiments, the present invention relates to the aforementioned kit, wherein said composition consists essentially of the polycation and the polyanion.

In certain embodiments, the present invention relates to the aforementioned kit, wherein said composition consists of the polycation and the polyanion.

In certain embodiments, the present invention relates to the aforementioned kit, wherein said composition is a solid at ambient temperature or physiological temperature.

In certain embodiments, the present invention relates to the aforementioned kit, wherein said composition further comprises fibrin, fibrionogen, polyvinyl alcohol, alginate or gellan.

In certain embodiments, the present invention relates to the aforementioned kit, wherein said composition further comprises fibrinogen.

In certain embodiments, the present invention relates to the aforementioned kit, wherein said composition further comprises thrombin, borate, boronate, calcium, or magnesium.

In certain embodiments, the present invention relates to the aforementioned kit, wherein said composition further comprises thrombin.

In certain embodiments, the present invention relates to the aforementioned kit, wherein said composition further comprises calcium chloride.

In certain embodiments, the present invention relates to the aforementioned kit, further comprising a second container comprising fibrin, fibrionogen, polyvinyl alcohol, alginate or gellan.

In certain embodiments, the present invention relates to the aforementioned kit, further comprising a second container comprising fibrionogen.

In certain embodiments, the present invention relates to the aforementioned kit, further comprising a second container comprising thrombin, borate, boronate, calcium, or magnesium.

In certain embodiments, the present invention relates to the aforementioned kit, further comprising a second container comprising thrombin.

In certain embodiments, the present invention relates to the aforementioned kit, wherein said polycation has a molecular weight greater than about 10 kD and less than about 500 kD.

In certain embodiments, the present invention relates to the aforementioned kit, wherein said polycation has a molecular weight greater than about 10 kD and less than about 250 kD.

In certain embodiments, the present invention relates to the aforementioned kit, wherein said polycation has a molecular weight greater than about 10 kD and less than about 200 kD.

In certain embodiments, the present invention relates to the aforementioned kit, wherein said polycation is a poly(amino acid).

In certain embodiments, the present invention relates to the aforementioned kit, wherein said polycation is a poly(amino acid); and said polycation contains at least about 50 amino acid residues and less than about 4000 amino acid residues.

In certain embodiments, the present invention relates to the aforementioned kit, wherein said polycation is a poly(amino acid); and said polycation contains at least about 100 amino acid residues and less than about 4000 amino acid residues.

In certain embodiments, the present invention relates to the aforementioned kit, wherein said polycation is a poly(amino acid); and said polycation contains at least about 200 amino acid residues and less than about 4000 amino acid residues.

In certain embodiments, the present invention relates to the aforementioned kit, wherein said polycation is a poly(amino acid); and said polycation contains at least about 300 amino acid residues and less than about 4000 amino acid residues.

In certain embodiments, the present invention relates to the aforementioned kit, wherein said polycation is a poly(amino acid); and said polycation contains at least about 500 amino acid residues and less than about 4000 amino acid residues.

In certain embodiments, the present invention relates to the aforementioned kit, wherein said polycation is a poly(amino acid); and said polycation contains at least about 750 amino acid residues and less than about 4000 amino acid residues.

In certain embodiments, the present invention relates to the aforementioned kit, wherein said polycation is a poly(amino acid); and said polycation contains at least about 1000 amino acid residues and less than about 4000 amino acid residues.

In certain embodiments, the present invention relates to the aforementioned kit, wherein said polycation is a poly(amino acid); and said polycation contains at least about 2000 amino acid residues and less than about 4000 amino acid residues.

In certain embodiments, the present invention relates to the aforementioned kit, wherein said polycation is a poly(amino acid); and said polycation contains at least about 3000 amino acid residues and less than about 4000 amino acid residues In certain embodiments, the present invention relates to the aforementioned kit, wherein said polycation is a poly(amino acid); said poly(amino acid) comprises a plurality of amino acids independently selected from the group consisting of Asp, Glu, Lys, Om, Arg, Gly, Ala, Val, Leu, Ile, Met, Pro, Phe, Trp, Asn, Gln, Ser, Thr, Tyr, Cys, and His; provided that no less than about twenty-five percent of the amino acids are independently selected from the group consisting of Lys, Om, His and Arg; further provided that no more than five percent of the amino acids are independently selected from the group consisting of Asp and Glu.

In certain embodiments, the present invention relates to the aforementioned kit, wherein said polycation is a poly(amino acid); said poly(amino acid) is represented by poly(X-Y), poly(X-Y-Y), or poly(X-Y-Y-Y); X is independently for each occurrence Lys, Orn, His or Arg; and Y is independently for each occurrence Gly, Ala, Val, Leu, Ile, Met, Pro, Phe, Trp, Asn, Gln, Ser, Thr, Tyr, or Cys.

In certain embodiments, the present invention relates to the aforementioned kit, wherein said polycation is a poly(amino acid); said poly(amino acid) is represented by poly(X-Y), poly(X-Y-Y), or poly(X-Y-Y-Y); X is Lys; and Y is independently for each occurrence Gly, Ala, Val, Leu, Ile, Met, Pro, Phe, Trp, Asn, Gln, Ser, Thr, Tyr, Cys, or His.

In certain embodiments, the present invention relates to the aforementioned kit, wherein said polycation is poly(Lys), poly(Orn), poly(Arg) and poly(His).

In certain embodiments, the present invention relates to the aforementioned kit, wherein said polycation is poly(L-Lys).

In certain embodiments, the present invention relates to the aforementioned kit, wherein said polycation is poly(Orn).

In certain embodiments, the present invention relates to the aforementioned kit, wherein said polycation degrades under physiological conditions in about 1 to about 12 weeks.

In certain embodiments, the present invention relates to the aforementioned kit, wherein said polycation degrades under physiological conditions in about 1 to about 6 weeks.

In certain embodiments, the present invention relates to the aforementioned kit, wherein said polycation degrades under physiological conditions in about 1 to about 4 weeks.

In certain embodiments, the present invention relates to the aforementioned kit, wherein said polycation degrades under physiological conditions in about 2 to about 5 weeks.

In certain embodiments, the present invention relates to the aforementioned kit, wherein said polyanion has a molecular weight greater than about 10 kD and less than about 500 kD.

In certain embodiments, the present invention relates to the aforementioned kit, wherein said polyanion has a molecular weight greater than about 20 kD and less than about 250 kD.

In certain embodiments, the present invention relates to the aforementioned kit, wherein said polyanion has a molecular weight greater than about 20 kD and less than about 100 kD.

In certain embodiments, the present invention relates to the aforementioned kit, wherein said polyanion is a poly(saccharide).

In certain embodiments, the present invention relates to the aforementioned kit, wherein said polyanion is a poly(saccharide); and said polyanion contains at least about 5 saccharide residues and less than about 2,500 saccharide residues.

In certain embodiments, the present invention relates to the aforementioned kit, wherein said polyanion is a poly(saccharide); and said polyanion contains at least about 20 saccharide residues and less than about 2,500 saccharide residues.

In certain embodiments, the present invention relates to the aforementioned kit, wherein said polyanion is a poly(saccharide); and said polyanion contains at least about 50 saccharide residues and less than about 2,500 saccharide residues.

In certain embodiments, the present invention relates to the aforementioned kit, wherein said polyanion is a poly(saccharide); and said polyanion contains at least about 100 saccharide residues and less than about 2,500 saccharide residues.

In certain embodiments, the present invention relates to the aforementioned kit, wherein said polyanion is a poly(saccharide); and said polyanion contains at least about 200 saccharide residues and less than about 2,500 saccharide residues.

In certain embodiments, the present invention relates to the aforementioned kit, wherein said polyanion is a poly(saccharide); and said polyanion contains at least about 300 saccharide residues and less than about 2,500 saccharide residues.

In certain embodiments, the present invention relates to the aforementioned kit, wherein said polyanion is a poly(saccharide); and said polyanion contains at least about 500 saccharide residues and less than about 2,500 saccharide residues.

In certain embodiments, the present invention relates to the aforementioned kit, wherein said polyanion is a poly(saccharide); and said polyanion contains at least about 750 saccharide residues and less than about 2,500 saccharide residues.

In certain embodiments, the present invention relates to the aforementioned kit, wherein said polyanion is a poly(saccharide); and said polyanion contains at least about 1,000 saccharide residues and less than about 2,500 saccharide residues.

In certain embodiments, the present invention relates to the aforementioned kit, wherein said polyanion is a poly(saccharide); and said polyanion contains at least about 1,500 saccharide residues and less than about 2,500 saccharide residues.

In certain embodiments, the present invention relates to the aforementioned kit, wherein said polyanion is a poly(saccharide); and said polyanion contains at least about 2,000 saccharide residues and less than about 2,500 saccharide residues.

In certain embodiments, the present invention relates to the aforementioned kit, wherein said polyanion is a poly(saccharide); and said saccharides are selected from the group consisting of cellulose, xylose, N-acetyllactosamine, glucuronic acid, mannuronic acid, and guluronic acid.

In certain embodiments, the present invention relates to the aforementioned kit, wherein said polyanion is a poly(saccharide); and a plurality of said saccharides are sulfated.

In certain embodiments, the present invention relates to the aforementioned kit, wherein said polyanion is a poly(saccharide); and a plurality of said saccharides are carboxymethylated.

In certain embodiments, the present invention relates to the aforementioned kit, wherein said polyanion is a poly(saccharide) selected from the group consisting of heparan sulfate, derrnatan sulfate, chondroitin sulfate, pentosan sulfate, keratan sulfate, mucopolysaccharide polysulfate, carrageenan, sodium alginate, potassium alginate, hyaluronic acid, and carboxymethylcellulose.

In certain embodiments, the present invention relates to the aforementioned kit, wherein said polyanion is chondroitin sulfate.

In certain embodiments, the present invention relates to the aforementioned kit, wherein said polyanion is a poly(amino acid).

In certain embodiments, the present invention relates to the aforementioned kit, wherein said polyanion is a poly(amino acid); and said polycation contains at least about 50 amino acid residues and less than about 4000 amino acid residues.

In certain embodiments, the present invention relates to the aforementioned kit, wherein said polyanion is a poly(amino acid); and said polycation contains at least about 100 amino acid residues and less than about 4000 amino acid residues.

In certain embodiments, the present invention relates to the aforementioned kit, wherein said polyanion is a poly(amino acid); and said polycation contains at least about 200 amino acid residues and less than about 4000 amino acid residues.

In certain embodiments, the present invention relates to the aforementioned kit, wherein said polyanion is a poly(amino acid); and said polycation contains at least about 300 amino acid residues and less than about 4000 amino acid residues.

In certain embodiments, the present invention relates to the aforementioned kit, wherein said polyanion is a poly(amino acid); and said polycation contains at least about 500 amino acid residues and less than about 4000 amino acid residues.

In certain embodiments, the present invention relates to the aforementioned kit, wherein said polyanion is a poly(amino acid); and said polycation contains at least about 750 amino acid residues and less than about 4000 amino acid residues.

In certain embodiments, the present invention relates to the aforementioned kit, wherein said polyanion is a poly(amino acid); and said polycation contains at least about 1000 amino acid residues and less than about 4000 amino acid residues.

In certain embodiments, the present invention relates to the aforementioned kit, wherein said polyanion is a poly(amino acid); and said polycation contains at least about 2000 amino acid residues and less than about 4000 amino acid residues.

In certain embodiments, the present invention relates to the aforementioned kit, wherein said polyanion is a poly(amino acid); and said polycation contains at least about 3000 amino acid residues and less than about 4000 amino acid residues In certain embodiments, the present invention relates to the aforementioned kit, wherein said polyanion is a poly(amino acid); said poly(amino acid) comprises a plurality of amino acids independently selected from the group consisting of Asp, Glu, Lys, Orn, Arg, Gly, Ala, Val, Leu, Ile, Met, Pro, Phe, Trp, Asn, Gln, Ser, Thr, Tyr, Cys, and His; provided that no less than about twenty-five percent of the amino acids are independently selected from the group consisting of Asp and Glu; further provided that no more than five percent of the amino acids are independently selected from the group consisting of Lys, Orn, and Arg.

In certain embodiments, the present invention relates to the aforementioned kit, wherein said polyanion is a poly(amino acid); said poly(amino acid) is represented by poly(X-Y), poly(X-Y-Y), or poly(X-Y-Y-Y); X is independently for each occurrence Asp or Glu; and Y is independently for each occurrence Gly, Ala, Val, Leu, Ile, Met, Pro, Phe, Trp, Asn, Gln, Ser, Thr, Tyr, Cys, or His.

In certain embodiments, the present invention relates to the aforementioned kit, wherein said polyanion is poly(Glu).

In certain embodiments, the present invention relates to the aforementioned kit, wherein said polyanion is poly(Asp).

In certain embodiments, the present invention relates to the aforementioned kit, wherein said polyanion degrades under physiological conditions in about 1 to about 12 weeks.

In certain embodiments, the present invention relates to the aforementioned kit, wherein said polyanion degrades under physiological conditions in about 1 to about 6 weeks.

In certain embodiments, the present invention relates to the aforementioned kit, wherein said polyanion degrades under physiological conditions in about 1 to about 4 weeks.

In certain embodiments, the present invention relates to the aforementioned kit, wherein said polyanion degrades under physiological conditions in about 2 to about 5 weeks.

In certain embodiments, the present invention relates to the aforementioned kit, wherein said first container further comprises an anti-infective; wherein said anti-infective is selected from the group consisting of an aminoglycoside, a tetracycline, a sulfonamide, p-aminobenzoic acid, a diaminopyrimidine, a quinolone, a β-lactam, a β-lactamase inhibitor, chloraphenicol, a macrolide, penicillins, cephalosporins, linomycin, clindamycin, spectinomycin, polymyxin B, colistin, vancomycin, bacitracin, isoniazid, rifampin, ethambutol, ethionamide, aminosalicylic acid, cycloserine, capreomycin, a sulfone, clofazimine, thalidomide, a polyene antifungal, flucytosine, imidazole, triazole, griseofulvin, terconazole, butoconazole ciclopirax, ciclopirox olamine, haloprogin, tolnaftate, naftifine, and terbinafine, or a combination thereof.

In certain embodiments, the present invention relates to the aforementioned kit, wherein said first container further comprises an anti-infective; wherein said anti-infective is tetracycline.

In certain embodiments, the present invention relates to the aforementioned kit, wherein said first container further comprises a contrast-enhancing agent.

In certain embodiments, the present invention relates to the aforementioned kit, wherein said first container further comprises a contrast-enhancing agent; wherein said contrast-enhancing agent is selected from the group consisting of radiopaque materials, paramagnetic materials, heavy atoms, transition metals, lanthanides, actinides, dyes, and radionuclide-containing materials.

Selacted Additional Therapeutic Applicants

In addition to being useful for treating emphysema (e.g., as described above and in the following examples), compositions of the invention may be used in other therapeutic applications.

By way of example only, any of a number of antibiotics and antimicrobials may be included in the hydrogels used in the methods of the invention. Antimicrobial drugs preferred for inclusion in compositions used in the methods of the invention include salts of lactam drugs, quinolone drugs, ciprofloxacin, norfloxacin, tetracycline, erythromycin, amikacin, triclosan, doxycycline, capreomycin, chlorhexidine, chlortetracycline, oxytetracycline, clindamycin, ethambutol, hexamidine isethionate, metronidazole, pentamidine, gentamicin, kanamycin, lineomycin, methacycline, methenamine,.minocycline, neomycin, netilmicin, paromomycin, streptomycin, tobramycin, miconazole and amanfadine and the like.

Another aspect of the invention may involve the use of a polycationic hydrogel composition to treat pleural effusions. Pleural effusions may be, for instance, ones that are refractory to medical therapy, such as malignant pleural effusions and benign, but recurrent, pleural effusions. Pleural effusions may be treated by methods such as administering a polycationic hydrogel composition within the pleural space to initiate sclerosis.

Another aspect of the invention involves the use of a polycationic hydrogel composition to treat post-operative and post traumatic wound bleeding. Wound bleeding may be treated by methods such as administering a polycationic hydrogel composition near the wound to inducing responses such as scarring.

Another aspect of the invention involves the use of a polycationic hydrogel composition to treat endoluminal bleeding. Examples of endoluminal bleeding include upper gastrointestinal bleeding from the esophagus or stomach, lower gastrointestinal bleeding from hemorrhoids or masses in the rectum or colon, and peritioneal bleeding from intraperitoneal cancers. Endoluminal bleeding may be treated by methods such as administering a polycationic hydrogel composition near and/or into the bleeding lesions to promote local microvascular thrombosis and/or rapid scar formation.

It should be appreciated that for all types of therapies the concentration of polycations to be used can be optimized experimentally. In addition, the duration of exposure and the type of polycationic hydrogel composition (e.g., its ability to induce a specific response in a targeted region) are important considerations. For certain applications, an appropriate polycation concentration may be chosen as one that results in 50% to 90% lysis (preferably about 80% lysis). The concentration required to induce lysis will depend, of course, on the type of cells in which the polycationic hydrogel compositions are exposed. Therefore, different diseases, which may occur in different regions of the body and which may be characterized by different cell types, may require different concentrations, amounts, or exposure times for one or more predetermined polycations in order to induce a desired response within a specific region of a patient. In some embodiments, the following in vitro assay can be used to determine appropriate concentrations of polycations. A flask of cells (e.g., fibroblast 3T3 cells, epithelial A549 cells, or other cells indicative of a targeted region of the body) is trypsinized and the cell suspension is split 1/10 and grown to about 80% confluence in a flask. A polycationic hydrogel composition (e.g., in the form of a solution, suspension, solid, or gel) can be added to this flask and left for about 2 minutes before being washed out. The polycation may be provided, for instance, in an isotonic salt solution. In one embodiment, the polycations are washed out (e.g., using an isotonic solution), and the percentage of lysed cells is evaluated. The cells may be stained using Trypan or another stain. The percentage of lysed cells may be calculated by comparing pictures of the flask surface (on which the cells were grown) before and after polycation exposure. The percentage lysis can be approximated by calculating the percentage of the flask surface that was cleared by the polycation. By testing different polycation concentrations, a concentration that produces the desired degree of lysis can be identified.

For many polycations, a range of concentrations may be effective. For example, in certain embodiments, between 0.25% and 2% poly-L-lysine may be used. However, other concentrations also may be used (e.g., 0.1% to 5.0%). Higher or lower concentrations may be used depending on the potency of the polycation, the time of exposure to the tissue, the rate of release of the polycation, the type of disease to be treated, etc. For example, a lower concentration may be used when a more potent polycation is used or when a longer exposure time is used. Certain polycations may be more potent when they have a higher molecular weight and/or a high charge density (i.e., higher number of charged groups).

The “potency” of a compound, as used herein, refers the ability of the compound to produce a desired result in a certain group of cells or in a target region of the body. In one aspect of the invention, the potency of a polycation refers to the ability of the polycation to produce a toxic effect on cells, such as cell death. In one particular embodiment, potency may be evaluated by growing cells on gels (e.g., split a cell suspension 1/10 and lay it on a 3% fibrinogen gel) that include different concentrations of one or more polycations. In some cases, the cells are then incubated for about 72 hours. At low concentrations, a polycation may facilitate cell attachment. However, at higher concentrations, a polycation may have a toxic response, i.e., the polycation may cause cells to round up and die. According to one aspect of the invention, polycation concentrations that have a toxic response and prevent cell growth and/or cause cells to die are chosen to be included in a polycationic hydrogel composition for treating a diseased patient. The toxic response will depend, of course, on the type of cells in which the polycationic hydrogel compositions are exposed. Therefore, different diseases, which may occur in different regions of the body and which may be characterized by different cell types, may require different concentrations of polycations in order to induce a desired response within a specific region of a patient.

### EXEMPLIFICATION

The invention now being generally described, it will be more readily understood by reference to the following examples, which are included merely for purposes of illustration of certain aspects and embodiments of the present invention, and are not intended to limit the invention.

### Example 1

### Complexation Behavior of Poly-L-Lysine with Various Polyanions

8 a] Polylysine+Chondroitin Sulfate: 50 mg of polylysine were dissolved in 5 mL of 50 mM Tris buffer, pH 7.4. To this solution, either 25 mg or 75 mg of chondroitin sulfate dissolved in 5 mL of 50 mM Tris buffer, pH 7.4, was added. An immediate precipitate formed. The supernatant was analyzed for polylysine content by RP-HPLC and less than about 0.1 mg/mL (limit of detection) was found in each case. These results indicate that more than 98% of polylysine can be precipitated by the addition of the polyanion chondroitin sulfate.

[b] Polylysine+Polyglutamic acid: The same experiment was repeated for polyglutamic acid as a polyanion with identical results.

[c] Polylysine+Alginate: The same experiment was repeated for alginate as a polyanion with identical results.

### Example 2

### Release of Complex from Hydrogels

To characterize the release characteristics of the complex of polylysine and chondrotitin sulfate from a fibrin hydrogel, the amount of poly-L-lysine released was assessed in vitro.

To 2 mL of a fibrinogen solution containing 65 mg/mL fibrinogen was added 4 mL of a 12.5 mg/mL chondroitin sulfate solution and after mixing, 2 mL of a 25 mg/mL polylysine solution was added. A precipitate was formed between polylysine and chondroitin sulfate. The solution was polymerized by the addition of 1,000 units of thrombin. Samples from a phosphate buffered saline extract of fibrin hydrogels were procured at 1, 2, 3, 6, 24 and 48 hours from polymerization and samples of extract were analyzed for polylysine by RP-HPLC. No polylysine was detected in the extracts at any of the time points evaluated.

The same experiment was repeated for a polyvinyl alcohol containing hydrogel formulation with the same relative amounts of polylysine and chondroitin sulfate. Samples from the phosphate buffered saline extract did not show any detectable polylysine by RP-HPLC.

### Example 3

### In-Vivo Experiments with Polylysine

Polylysine is a known fibrosing agent and was used to induce local lung injury and to induce scarring and fibrosis leading to lung volume reduction.

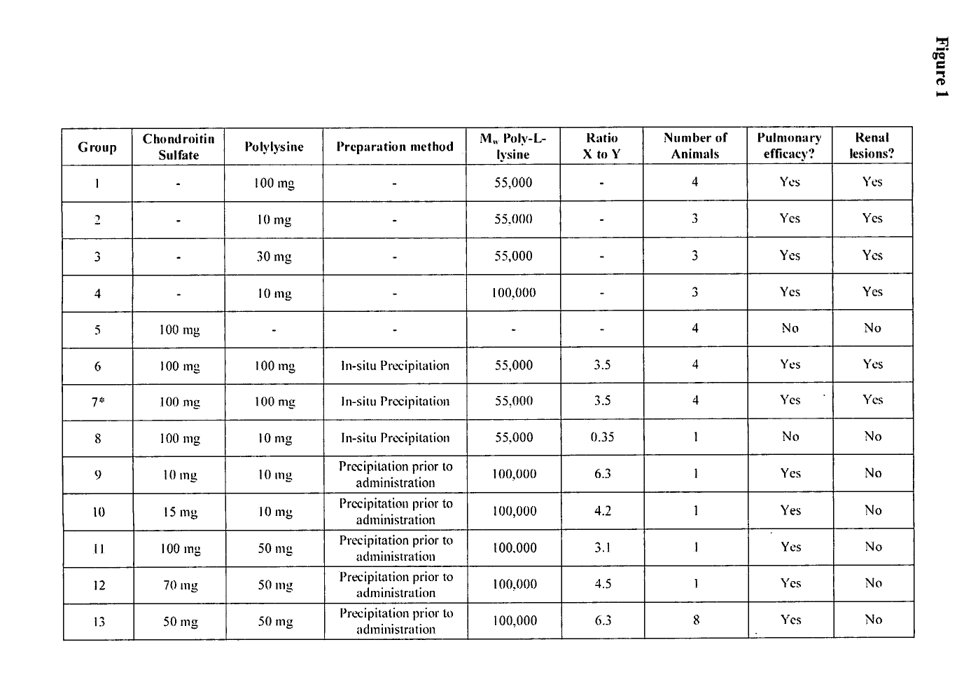

Polylysine was delivered to 12 pulmonary subsegments via a bronchoscope in a fibrin gel matrix at 100 mg, 30 mg and 10 mg per subsegment (groups 1, 2 and 3 in FIG. 1). The molecular weight of the polylysine used was 20,000-80,000 Da with an average molecular weight of 55,000 Da. A total of 10 sheep were thus treated.

Treatments containing 100 mg/treatment of polylysine caused renal toxicity manifest as nephropathy and infarction as well as severe lung injury. Preparations containing 10 and 30 mg/treatment of polylysine produced acceptable pulmonary responses, but were associated with renal toxicity.

In a total of 3 sheep (Group 4 in FIG. 1), using high molecular weight polylysine (80,000-130,000 Da, average molecular weight 100,000) in place of lower molecular weight PLL also did not prevent renal toxicity.

The characteristic lesion caused by polylysine is renal infarction, consistent with what has been reported in the literature for polycationic injury. In the animals tested with baseline normal renal function, renal damage resulting from cationic injury remains subclinical. No abnormalities in serum BUN or creatinine, or in urine analysis or urine protein-to-creatinine ratio were observed in this study among animals with renal lesions at necropsy. Thus, these clinical pathology tests were not sufficiently sensitive to detect polycationic renal injury resulting from polylysine. Polycationic renal toxicity following pulmonary treatment is initiated and can be detected only at necropsy within days of treatment. All animals tested in the study with formulations containing polycationic material alone displayed gross evidence of large renal lesions at the time of necropsy. The characteristic acute lesion appeared to be renal infarction with associated hemorrhage. Lesions occurred within days (3-7 days) of cationic exposure. Necropsy findings are the most sensitive markers of toxicity.

### Example 4

### In-vivo Experiments with the Polyanion Chondroitin Sulfate

4 sheep were treated at 12 pulmonary subsegments each with a fibrin gel containing 100 mg chondroitin sulfate (Group 5 in FIG. 1). 2 animals were sacrificed at 8 days and the remaining 2 animals were sacrificed at 4 weeks. Animals receiving treatments containing only chondroitin sulfate instead of polylysine had no renal lesions. However, pulmonary responses in these animals were minimal, indicating poor efficacy.

These experiments establish that polylysine is the specific substance responsible for the local toxicity desired but also responsible for the systemic renal toxicity. This conclusion is based upon observations showing that preparations without polylysine were associated with no renal toxicity, but no pulmonary efficiency, while all preparations containing free polylysine, involving a broad range of concentrations, were associated with pulmonary efficiency and renal lesions.

### Example 5

### In-Vivo Experiments with In-Situ Precipitation of Poly-L-Lysine & Chondroitin Sulfate

In a series of 4 sheep experiments (Group 6 in FIG. 1), in-situ precipitation of chondroitin sulfate and polylysine solutions was achieved as they exit a dual lumen catheter. Chondroitin sulfate was added to a fibrinogen solution to a final concentration of 20 mg/mL, while polylysine was added to a thrombin solution to a final concentration of 20 mg/mL. Both solutions were 5 mL and were injected through a dual lumen catheter into subsegments of sheep lungs for a total of 10 mL per subsegment.

Excellent pulmonary response was found with lung volume reduction exhibited in the treated sheep. However, renal lesions were found and the in-situ precipitation method of mediating the systemic toxicity of polylysine while preserving the local toxicity was unsuccessful.

In a total of 4 sheep (Group 7 in FIG. 1), use of systemic heparin to complex polylysine in the circulation and reduce or prevent renal toxicity was attempted. It did not prevent renal lesions at the doses tested.

A tenfold reduction in polylysine content relative to chondroitin sulfate content (Group 8 in FIG. 1) prevented the occurrence of renal lesions, but eliminated pulmonary responses.

### Example 6

### In-Vivo Experiments with Precipitated Polylysine/Chondroitin Sulfate

Precipitation of chondroitin sulfate and polylysine in the fibrinogen solution appeared to eliminate renal toxicity as detected by the presence of gross renal lesions at necropsy. Rapid, complete polymerization of a 9:1.4 fibrinogen:thrombin preparation could be accomplished utilizing various ratios of chondroitin sulfate to polylysine.

Precipitation of polylysine with chondroitin sulfate in 10 mL of fibrinogen solution and polymerization with 1000 units of thrombin prevented the occurrence of renal infarction. Using this precipitation methodology, polylysine related renal toxicity was prevented over a broad polylysine concentration range from 1 mg/mL to 10 mg/mL at chondroitin sulfate concentration of 1 to 5 mg/mL (Groups 9 to 13 in FIG. 1).

Lung volume reduction treatment was performed in 8 consecutive animals at 84 subseguential sites using a formulation containing 13 mg/mL of human fibrinogen, 5 mg/mL of sodium chondroitin sulfate, 5 mg/mL of poly-L-lysine, and polymerized in situ with 1000 U activated human thrombin (Group 13 in FIG. 1). This treatment produced contracted pulmonary lesions without evidence of unexpected local tissue toxicity, and without evidence of renal toxicity.

### Example 7

### In-Vivo Experiments with Poly(Lys, Glu)

The precipitation of polylysine with chondroitin sulfate might be mimicked by incorporating the negative charge into a copolymer, thereby reducing the complexity of the system. This approach was tested with a copolymer of lysine and glutamic acid (MW 150,000-300,000; ratio Lys:Glu 4:1).

Three rats were treated with 5 mg/mL poly(Lys, Glu) to evaluate whether the copolymer could be used to modulate local and/or systemic toxicity. Treatments contained 28.6 mg/mL fibrinogen polymerized with 200 U/mL thrombin. All rats were anesthetized and intubated orotracheally. A dual lumen catheter was placed into a target site in the lung with bronchoscopic guidance. The reagents were injected into the lung and the catheter was removed. Each animal was allowed to recover from anesthesia, and returned to its cage. After 1 week, all animals were euthanized. The extent of pleural scarring was assessed prior to lung removal from the chest cavity. The lungs were then removed en bloc, fully inflated, and evaluated visually to assess the extent of local parenchymal inflammation and scarring produced by treatment. The kidneys were also harvested and evaluated for the presence of cortical lesions and infarctions which can develop as a consequence of systemic toxicity following polycation adminstration.

Rats that received the copolymer demonstrated no significant local pulmonary lesions, and no incidence of systemic toxicity, manifest as renal lesions.

The results indicated that the copolymer consisting of both cationic and anionic segments did not show any systemic toxicity, but failed to demonstrate efficacy as a lung volume reduction agent.

### Example 8

### In-Vivo Experiments with Polyornithine

Rats underwent lung volume reduction with polyornithine.

4 rats were treated with 2.5 mg/mL polyomithine and 3 were treated with 2.5 mg/mL polyomithine precipitated with 2.5 mg/mL chondroitin sulfate to evaluate whether precipitation could be used to modulate local and/or systemic toxicity. Treatments contained 28.6 mg/mL fibrinogen polymerized with 200 U/mL thrombin. All rats were anesthetized and intubated orotracheally. A dual lumen catheter was placed into a target site in the lung with bronchoscopic guidance. The reagents were injected into the lung and the catheter was removed. Each animal was allowed to recover from anesthesia, and returned to its cage. After 1 week, all animals were euthanized. The extent of pleural scarring was assessed prior to lung removal from the chest cavity. The lungs were then removed en bloc, fully inflated, and evaluated visually to assess the extent of local parenchymal inflammation and scarring produced by treatment. The kidneys were also harvested and evaluated for the presence of cortical lesions and infarctions which can develop as a consequence of systemic toxicity following polycation administration.

Rats that received polyornithine demonstrated significant local toxicity, and systemic toxicity, manifest as renal lesions. In the rats that received precipitated polyornithine, the incidence of renal leasons was decreased.

The results indicated that precipitating polycations with a polyanion significantly decreased the severity of local injury and incidence of systemic toxicity.

### Example 9

### In-Vivo Experiments with PVA/Borate

The safety and effectiveness of precipitated polycations/polyanions for the purpose of producing local scarring, contraction, and volume reduction in the lung has also been tested using non-fibrin hydrogel systems. 4% Polyvinylalcohol (PVA) containing 5 mg/mL poly-L-Lysine precipitated with 5 mg/mL chondroitin sulfate polymerized with 4% sodium borate has been evaluated. Treatments were administered endobronchially at twelve subsegmental sites in healthy experimentally naive sheep following administration of anesthesia, fiberoptic intubation, and initiation of mechanical ventilator support.

PVA was evaluated in 6 sheep administered either as a foam in which PVA gel is combined with oxygen (in 3 animals), or directly as a gel (in 3 animals). Results are available out to 1 week at which time therapeutic safety was evaluated by necropsy assessment of treatment sites and vital organs, and effectiveness was assessed by radiographic assessment of treatment-related changes in lung volumes and necropsy assessment of pulmonary responses.

Results showed that the precipitated polylysine/chondroitin sulfate in PVA foams or gels caused effective volume reduction associated with localized areas of lung collapse at sites of treatment. Ten mL injections of PVA treatment, administered either as a foam or gel were associated with a 0.7-1.2% volume reduction/site. None of the six animals tested had evidence of systemic toxicity. Specifically, there was no necropsy evidence of renal, hepatic, cardiac, adrenal, or splenic lesions.

These data indicate that precipitation of polycations such as polylysine to polyanions such as chondroitin sulfate can be achieved in hydrogel systems other than fibrin-gels and delivered in vivo to affect localized tissue injury and achieving lung volume reduction without causing systemic toxicity.

### Example 10

### Testing of Poly-L-lysine/Chondroitin Sulfate Complexes in Emphysema Patients to Achieve Lung Volume Reduction.

A system for producing controlled, localized tissue injury using a polycation complexed to a polyanion for the purpose of achieving lung volume reduction in patients with advanced emphysema was developed and completed initial clinical testing. In this formulation, a suspension containing 13 mg/mL of human fibrinogen, 0.5 mg/mL of aqueous tetracycline hydrochloride, 5 mg/mL of poly-L-lysine acetate, and 5 mg/mL of chondroitin sulfate was administered simultaneously with a calcium chloride solution containing 1500 units of human thrombin endobronchial through a catheter positioned within the airway using a flexible bronchoscope. The fibrinogen-thrombin mixture polymerized in-situ to generate a gel at the site of treatment. The precipitated polylysine/chondroitin sulfate caused a localized injury, which collapses and scars the damaged area of lung.

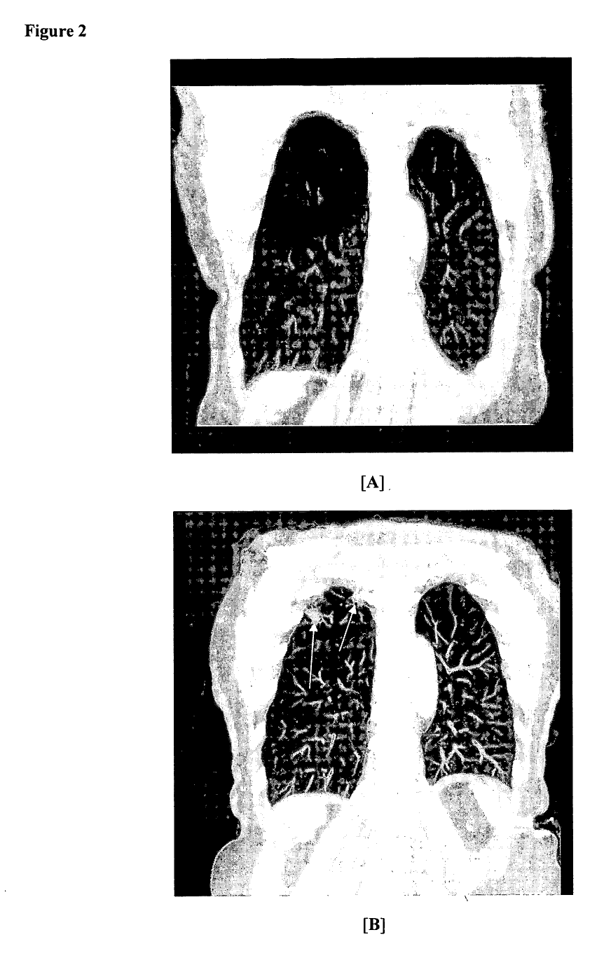

Six patients were treated at 4 subsegmental airway sites in a single lung using this formulation. Chest CT images performed at 6 weeks showed evidence of localized scarring at sites of treatment. Examples of scar formation are shown in the CT images in FIG. 2.

Physiological measurements showed reductions in lung volumes (RV/TLC ratio decreased an average of 4%) and improvements in vital capacity (increased an average of 13%), both of which are considered clinically significant and compare well to unilateral lung volume reduction surgery.

Renal ultrasounds were performed at baseline prior to treatment, at 1 day post-treatment, and at 1 week post-treatment to assess for possible renal toxicity that can be caused by polycationic injury. Blood urea nitrogen (BUN) and serum creatinine levels, and urine analysis were assessed at baseline, 1 day, and 1 week post-treatment to assess for changes in renal function or evidence of renal tissue damage. Renal ultrasound studies showed no evidence of post-treatment changes to indicate polycation injury in the form of renal infarction. Renal function studies, including BUN and creatinine, were not adversely affected at 1 day or 1 week post treatment. Urine analysis studies showed no evidence of renal injury. Additional clinical pathology testing further demonstrated there was no evidence of adverse effects of treatment on the cardiac, hepatic, or hematological systems.

These results confirmed that the polycation poly-L-lysine can be safely administered in a fibrin hydrogel to the lung of patients with emphysema to produce therapeutic lung volume reduction when precipitated with a polyanion (in this instance chondroitin sulfate) prior to administration of the hydrogel.

### INCORPORATION BY REFERENCE

U.S. Pat. No. 6,610,043, U.S. Pat. No. 6,709,401, U.S. Pat. No. 6,682,520, U.S. Patent Application 2002/0147462, U.S. Patent Application 2003/0018351, U.S. Patent Application 2003/0228344, U.S. Patent Application 2004/0200484, US Patent Application 2004/0038868, and U.S. Patent Application 2005/0239685 are all hereby incorporated by reference in their entirety. In addition, all of the US Patents and US Published Patent Applications cited herein are hereby incorporated by reference.

### Equivalents

While several embodiments of the present invention are described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

We claim:

1. A composition comprising a polycation and a polyanion; wherein the ratio of X to Y is greater than about one; X is the product of the mass of the polycation and the charge-per-mass ratio of the polycation; and Y is the product of the mass of the polyanion and the change-per-mass ratio of the polyanion.

2. The composition of claim 1, wherein said composition consists essentially of the polycation and the polyanion.

3. The composition of claim 1, wherein said composition consists of the polycation and the polyanion.

4. The composition of claim 1, wherein said composition is a solid at ambient temperature or physiological temperature.

5. The composition of claim 1, further comprising fibrin, fibrinogen, polyvinyl alcohol, alginate or gellan.

6. The composition of claim 1, further comprising fibrinogen.

7. The composition of claim 1, further comprising thrombin, borate, boronate, calcium, or magnesium.

8. The composition of claim 1, further comprising thrombin.

9. The composition of claim 1, further comprising calcium chloride.

10. The composition of claim 1, further comprising a hydrogel formed from the combination of fibrin and thrombin; fibrinogen and thrombin; polyvinyl alcohol and borate; polyvinyl alcohol and a boronate; alginate and calcium; or gellan and magnesium.

11. The composition of claim 1, further comprising a hydrogel formed from the combination of fibrinogen and thrombin.

12. The composition of claim 1, wherein said polycation has a molecular weight greater than about 10 kD and less than about 500 kD.

13. The composition of claim 1, wherein said polycation has a molecular weight greater than about 10 kD and less than about 250 kD.

14. The composition of claim 1, wherein said polycation has a molecular weight greater than about 10 kD and less than about 200 kD.

15. The composition of claim 1, wherein said polycation is a poly(amino acid).

16. The composition of claim 1, wherein said polycation is a poly(amino acid); and said polycation contains at least about 50 amino acid residues and less than about 4000 amino acid residues.

17. The composition of claim 1, wherein said polycation is a poly(amino acid); and said polycation contains at least about 100 amino acid residues and less than about 4000 amino acid residues.

18. The composition of claim 1, wherein said polycation is a poly(amino acid); and said polycation contains at least about 200 amino acid residues and less than about 4000 amino acid residues.

19. The composition of claim 1, wherein said polycation is a poly(amino acid); and said polycation contains at least about 300 amino acid residues and less than about 4000 amino acid residues.

20. The composition of claim 1, wherein said polycation is a poly(amino acid); and said polycation contains at least about 500 amino acid residues and less than about 4000 amino acid residues.

21. The composition of claim 1, wherein said polycation is a poly(amino acid); and said polycation contains at least about 750 amino acid residues and less than about 4000 amino acid residues.

22. The composition of claim 1, wherein said polycation is a poly(amino acid); and said polycation contains at least about 1000 amino acid residues and less than about 4000 amino acid residues.

23. The composition of claim 1, wherein said polycation is a poly(amino acid); and said polycation contains at least about 2000 amino acid residues and less than about 4000 amino acid residues.

24. The composition of claim 1, wherein said polycation is a poly(amino acid); and said polycation contains at least about 3000 amino acid residues and less than about 4000 amino acid residues.

25. The composition of claim 1, wherein said polycation is a poly(amino acid); said poly(amino acid) comprises a plurality of amino acids independently selected from the group consisting of Asp, Glu, Lys, Om, Arg, Gly, Ala, Val, Leu, Ile, Met, Pro, Phe, Trp, Asn, Gln, Ser, Thr, Tyr, Cys, and His; provided that no less than about twenty-five percent of the amino acids are independently selected from the group consisting of Lys, Orn, His and Arg; further provided that no more than five percent of the amino acids are independently selected from the group consisting of Asp and Glu.

26. The composition of claim 1, wherein said polycation is a poly(amino acid); said poly(amino acid) is represented by poly(X-Y), poly(X-Y-Y), or poly(X-Y-Y-Y); X is independently for each occurrence Lys, Om, His or Arg; and Y is independently for each occurrence Gly, Ala, Val, Leu, Ile, Met, Pro, Phe, Trp, Asn, Gln, Ser, Thr, Tyr, or Cys.

27. The composition of claim 1, wherein said polycation is a poly(amino acid); said poly(amino acid) is represented by poly(X-Y), poly(X-Y-Y), or poly(X-Y-Y-Y); X is Lys; and Y is independently for each occurrence Gly, Ala, Val, Leu, Ile, Met, Pro, Phe, Trp, Asn, Gln, Ser, Thr, Tyr, Cys, or His.

28. The composition of claim 1, wherein said polycation is poly(Lys), poly(Orn), poly(Arg) or poly(His).

29. The composition of claim 1, wherein said polycation is poly(Lys).

30. The composition of claim 1, wherein said polycation is poly(L-Lys).

31. The composition of claim 1, wherein said polycation degrades under physiological conditions in about 1 to about 12 weeks.

32. The composition of claim 1, wherein said polycation degrades under physiological conditions in about 1 to about 6 weeks.

33. The composition of claim 1, wherein said polycation degrades under physiological conditions in about 1 to about 4 weeks.

34. The composition of claim 1, wherein said polycation degrades under physiological conditions in about 2 to about 5 weeks.

35. The composition of claim 1, wherein said polyanion has a molecular weight greater than about 10 kD and less than about 500 kD.

36. The composition of claim 1, wherein said polyanion has a molecular weight greater than about 20 kD and less than about 250 kD.

37. The composition of claim 1, wherein said polyanion has a molecular weight greater than about 20 kD and less than about 100 kD.

38. The composition of claim 1, wherein said polyanion is a poly(saccharide).

39. The composition of claim 1, wherein said polyanion is a poly(saccharide); and said polyanion contains at least about 5 saccharide residues and less than about 2,500 saccharide residues.

40. The composition of claim 1, wherein said polyanion is a poly(saccharide); and said polyanion contains at least about 20 saccharide residues and less than about 2,500 saccharide residues.

41. The composition of claim 1, wherein said polyanion is a poly(saccharide); and said polyanion contains at least about 50 saccharide residues and less than about 2,500 saccharide residues.

42. The composition of claim 1, wherein said polyanion is a poly(saccharide); and said polyanion contains at least about 100 saccharide residues and less than about 2,500 saccharide residues.

43. The composition of claim 1, wherein said polyanion is a poly(saccharide); and said polyanion contains at least about 200 saccharide residues and less than about 2,500 saccharide residues.

44. The composition of claim 1, wherein said polyanion is a poly(saccharide); and said polyanion contains at least about 300 saccharide residues and less than about 2,500 saccharide residues.

45. The composition of claim 1, wherein said polyanion is a poly(saccharide); and said polyanion contains at least about 500 saccharide residues and less than about 2,500 saccharide residues.

46. The composition of claim 1, wherein said polyanion is a poly(saccharide); and said polyanion contains at least about 750 saccharide residues and less than about 2,500 saccharide residues.

47. The composition of claim 1, wherein said polyanion is a poly(saccharide); and said polyanion contains at least about 1,000 saccharide residues and less than about 2,500 saccharide residues.

48. The composition of claim 1, wherein said polyanion is a poly(saccharide); and said polyanion contains at least about 1,500 saccharide residues and less than about 2,500 saccharide residues.

49. The composition of claim 1, wherein said polyanion is a poly(saccharide); and said polyanion contains at least about 2,000 saccharide residues and less than about 2,500 saccharide residues.

50. The composition of claim 1, wherein said polyanion is a poly(saccharide); and said saccharides are selected from the group consisting of cellulose, xylose, N-acetyllactosamine, glucuronic acid, mannuronic acid, and guluronic acid.

51. The composition of claim 1, wherein said polyanion is a poly(saccharide); and a plurality of said saccharides are sulfated.

52. The-composition of claim 1, wherein said polyanion is a poly(saccharide); and a plurality of said saccharides are carboxymethylated.

53. The composition of claim 1, wherein said polyanion is a poly(saccharide) selected from the group consisting of heparan sulfate, dermatan sulfate, chondroitin sulfate, pentosan sulfate, keratan sulfate, mucopolysaccharide polysulfate, carrageenan, sodium alginate, potassium alginate, hyaluronic acid, and carboxymethylcellulose.

54. The composition of claim 1, wherein said polyanion is chondroitin sulfate.

55. The composition of claim 1, wherein said polyanion is a poly(amino acid).

56. The composition of claim 1, wherein said polyanion is a poly(amino acid); and said polycation contains at least about 50 amino acid residues and less than about 4000 amino acid residues.

57. The composition of claim 1, wherein said polyanion is a poly(amino acid); and said polycation contains at least about 100 amino acid residues and less than about 4000 amino acid residues.

58. The composition of claim 1, wherein said polyanion is a poly(amino acid); and said polycation contains at least about 200 amino acid residues and less than about 4000 amino acid residues.

59. The composition of claim 1, wherein said polyanion is a poly(amino acid); and said polycation contains at least about 300 amino acid residues and less than about 4000 amino acid residues.

60. The composition of claim 1, wherein said polyanion is a poly(amino acid); and said polycation contains at least about 500 amino acid residues and less than about 4000 amino acid residues.

61. The composition of claim 1, wherein said polyanion is a poly(amino acid); and said polycation contains at least about 750 amino acid residues and less than about 4000 amino acid residues.

62. The composition of claim 1, wherein said polyanion is a poly(amino acid); and said polycation contains at least about 1000 amino acid residues and less than about 4000 amino acid residues.

63. The composition of claim 1, wherein said polyanion is a poly(amino acid); and said polycation contains at least about 2000 amino acid residues and less than about 4000 amino acid residues.

64. The composition of claim 1, wherein said polyanion is a poly(amino acid); and said polycation contains at least about 3000 amino acid residues and less than about 4000 amino acid residues.

65. The composition of claim 1, wherein said polyanion is a poly(amino acid); said poly(amino acid) comprises a plurality of amino acids independently selected from the group consisting of Asp, Glu, Lys, Om, Arg, Gly, Ala, Val, Leu, Ile, Met, Pro, Phe, Trp, Asn, Gln, Ser, Thr, Tyr, Cys, and His; provided that no less than about twenty-five percent of the amino acids are independently selected from the group consisting of Asp and Glu; further provided that no more than five percent of the amino acids are independently selected from the group consisting of Lys, Om, and Arg.

66. The composition of claim 1, wherein said polyanion is a poly(amino acid); said poly(amino acid) is represented by poly(X-Y), poly(X-Y-Y), or poly(X-Y-Y-Y); X is independently for each occurrence Asp or Glu; and Y is independently for each occurrence Gly, Ala, Val, Leu, Ile, Met, Pro, Phe, Trp, Asn, Gln, Ser, Thr, Tyr, Cys, or His.

67. The composition of claim 1, wherein said polyanion is poly(Glu).

68. The composition of claim 1, wherein said polyanion is poly(Asp).

69. The composition of claim 1, wherein said polyanion degrades under physiological conditions in about 1 to about 12 weeks.

70. The composition of claim 1, wherein said polyanion degrades under physiological conditions in about 1 to about 6 weeks.

71. The composition of claim 1, wherein said polyanion degrades under physiological conditions in about 1 to about 4 weeks.

72. The composition of claim 1, wherein said polyanion degrades under physiological conditions in about 2 to about 5 weeks.

73. The composition of claim 1, further comprising an anti-infective; wherein said anti-infective is selected from the group consisting of an aminoglycoside, a tetracycline, a sulfonamide, p-aminobenzoic acid, a diaminopyrimidine, a quinolone, a β-lactam, a β-lactamase inhibitor, chloraphenicol, a macrolide, penicillins, cephalosporins, linomycin, clindamycin, spectinomycin, polymyxin B, colistin, vancomycin, bacitracin, isoniazid, rifampin, ethambutol, ethionamide, aminosalicylic acid, cycloserine, capreomycin, a sulfone, clofazimine, thalidomide, a polyene antifungal, flucytosine, imidazole, triazole, griseofulvin, terconazole, butoconazole ciclopirax, ciclopirox olamine, haloprogin, tolnaftate, naftifine, and terbinafine, or a combination thereof.

74. The composition of claim 1, further comprising an anti-infective; wherein said anti-infective is tetracycline.

75. The composition of claim 1, further comprising a contrast-enhancing agent.

76. The composition of claim 1, further comprising a contrast-enhancing agent; wherein said contrast-enhancing agent is selected from the group consisting of radiopaque materials, paramagnetic materials, heavy atoms, transition metals, lanthanides, actinides, dyes, and radionuclide-containing materials.
